(19) United States
(12) Patent Application Publication
(10) Pub. No.: US 2024/0182874 A1
(43) Pub. Date: Jun. 6, 2024

(54) NOVEL FAMILY OF DNA POLYMERASES ACCEPTING 2-AMINOADENINE AND REJECTING ADENINE IN THEIR SUBSTRATES

(71) Applicants: THE EUROPEAN SYNDICATE OF SYNTHETIC SCIENTISTS AND INDUSTRIALISTS, Paris (FR); COMMISSARIAT À L´ÉNERGIE ATOMIQUE ET AUX ÉNERGIES ALTERNATIVES, Paris (FR)

(72) Inventors: Pierre-Yves Vincent BOURGUIGNON, Berlin (DE); Philippe MARLIÈRE, Luxembourg (LU); Valérie PEZO, Evry cedex (FR); Raphael MEHEUST, Evry cedex (FR)

(21) Appl. No.: 18/286,072

(22) PCT Filed: Apr. 13, 2022
(86) PCT No.: PCT/EP2022/059852

§ 371 Date: Oct. 6, 2023

(30) Foreign Application Priority Data
Apr. 15, 2021 (EP) 21 168 699.3

(51) Int. Cl. C12N 9/12; C12N 15/63; C12P 19/34

(52) U.S. Cl. CPC C12N 9/1252; C12N 15/63; C12P 19/34; C12Y 207/07007

(57) ABSTRACT

Described is a novel family of DNA polymerases identified in bacteriophages (mainly from the family of Siphiroviridae) which are able to accept 2-amino-2′-deoxyadenosine 5′-triphosphate (dZTP) as a substrate but which do not accept deoxyadenosine 5′-triphosphate (dATP) as a substrate. Also described are recombinant nucleic acid molecules, encoding such polymerases, vectors comprising such nucleic acid molecules, host cells transformed with such nucleic acid molecules or vectors as well as to methods for producing DNA molecules containing 2-amino-2′-deoxyadenosine (dZ) instead of 2′-deoxyadenosine (dA) by making use of a novel polymerase.

The present invention relates to a novel family of DNA polymerases identified in bacteriophages (mainly from the family of Siphoviridae) which are able to accept 2-amino-2′-deoxyadenosine 5′-triphosphate (dZTP) as a substrate but which do not accept deoxyadenosine 5′-triphosphate (dATP) as a substrate. The present invention in particular relates to recombinant nucleic acid molecules, encoding such polymerases, vectors comprising such nucleic acid molecules, host cells transformed with such nucleic acid molecules or vectors as well as to methods for producing DNA molecules containing 2-amino-2′-deoxyadenosine (dZ) instead of 2′-deoxyadenosine (dA) by making use of a novel polymerase.

Biology is defined as the study of extant and extinct species that have appeared during evolution (Mayr Ernst, This Is Biology (Belknap Press, 1998); Woese et al., Proceedings of the National Academy of Sciences. 87, 4576-4579 (1990)). In all generality, it also encompasses the viable yet virtual species that remain to emerge by means of natural selection or rational construction (Wong, Front Biosci. 19, 1117 (2014); Marliere, Syst Synth Biol. 3, 77-84 (2009)). The task to design, assemble and evolve virtual organisms pertains to synthetic biology (Benner and Sismour, Nat Rev Genet. 6, 533-543 (2005)) and more pointedly to xenobiology (Schmidt, Bioessays. 32, 322-331 (2010)). In this respect, one focus in evolving virtual organisms is the development of genetic material which is different from that which normally occurs in nature.

Until now, no genetic polymer other than DNA and RNA has been found in any living organism (Pochet et al., C. R. Biol. 326, 1175-1184 (2003); Pinheiro et al., Science. 336, 341-344 (2012)). In its totality, RNA transcribed or replicated by any cellular or viral species is believed to originate from polymerization of the four canonical monomers rATP, rCTP, rGTP, rUTP. By contrast, the chemical structure of DNA precursors seems to have undergone a wider diversification (Warren, Annu. Rev. Microbiol. 34, 137-158 (1980)). Surrogates of dNTPs bearing an altered pyrimidine or purine heterocycle were found to replace each of the canonical dATP, dCTP, dGTP or dTTP during replication of certain DNA viruses infecting bacteria (Warren, Annu. Rev. Microbiol. 34, 137-158 (1980); Weigele and Raleigh, Chem. Rev. 116, 12655-12687 (2016)). No species is known to replicate by substituting more than one canonical dNTP. Cellular genomes consist of DNA resulting exclusively from polymerization of the four canonical dNTPs (Marliere et al., Angew. Chem. Int. Ed. Engl. 50, 7109-7114 (2011); Mehta et al., J. Am. Chem. Soc. 138, 14230-14233 (2016)).

One of the most extreme chemical deviation reached during the evolution of natural species consists of the complete replacement of adenine (A) by 2-aminoadenine (also known as 2,6-diaminopurine and herein also referred to as Z) in the DNA from the lytic phage S-2L, a siphovirus which preys on freshwater cyanobacteria of the genus Synechococcus (Khudyakov et al., Virology. 88, 8-18 (1978)). This phage, whose double-stranded linear DNA contains about 35% G, 35% C, 15% T and 15% of the base Z, was discovered in 1977 (Kirnos et al., Nature. 270, 369-370 (1977)). Like the G:C pair, the Z:T pair is stabilized by three hydrogen-bonds, instead of two in A:T (Bailly, Nucleic Acids Research. 26, 4309-4314 (1998)), thus singularly violating canonical Watson-Crick pairing.

Nevertheless, the presence of Z in templates does not preclude the incorporation of dTTP by DNA polymerases (Bailly et al., Proceedings of the National Academy of Sciences. 93, 13623-13628 (1996)) and, thus, Z-encoded genetic information can be retrieved under the form of A-containing, canonical DNA through replication. It was this faithful transliteration of Z into A during bacterial cloning procedures which made it possible to elucidate the 45 kbp genome sequence of the phage S-2L in 1998 (Weigele et al., Chem. Rev. 116, 12655-12687 (2016); WO2003093461).

The phage genome displayed a gene, designated purZ, for a protein distantly related to succinoadenylate synthase (EC 6.3.4.4) the cellular enzyme of adenine biosynthesis, encoded by purA (WO2003093461). This finding pointed to a possibly existing phage-encoded metabolic pathway converting guanine deoxynucleotides into dZTP (2-amino-2′-deoxyadenosine 5′-triphosphate) which may constitute a putative DNA polymerase substrate. However, so far no gene encoding a DNA polymerase could be detected in the S-2L genome when it was sequenced (Genbank AX955019.1).

The identification of a polymerase which would, e.g., be able to selectively incorporate 2-amino-2′-deoxyadenosine 5′-triphosphate instead of 2′-deoxyadenosine 5′-triphosphate (dATP) into DNA molecules would provide a useful tool for developing virtual organisms. Thus, there was a need to verify whether the occurrence of the nucleotide Z in the genome of the S-2L is indeed due to the selective incorporation of dZTP by a polymerase into a growing polynucleotide chain or is based on another mechanism, such as selective removal or degradation of dATP prior to replication which thus favors the presence and consequently the incorporation of dZTP into growing polynucleotide chains or the favored excision of incorporated dA by a 3′-exonuclease activity in comparison to the excision of dZ.

This need is addressed by the provision of the embodiments as characterized in the claims.

In particular, the present inventors were able to identify a new functional category of polymerases which are able of using 2-amino-2′-deoxyadenosine 5′-triphosphate (dZTP) as a substrate and of incorporating it into a polynucleotide molecule and which, at the same time reject dATP. Thus, such polymerases are able to discriminate, as regards the base, for 2-aminoadenine and against adenine.

Thus, in a first aspect, the present invention relates to a recombinant nucleic acid molecule comprising a nucleotide sequence encoding a polymerase wherein the polymerase is characterized by the following features:
(a) it has an amino acid sequence which is at least 50% identical to the amino acid sequence shown in any one of SEQ ID NOs: 1 to 233;
(b) it does not show 5′-exonuclease activity;
(c) it accepts 2-amino-2′-deoxyadenosine 5′-triphosphate (dZTP) as a substrate; and
(d) it does not accept deoxyadenosine 5′-triphosphate (dATP) as a substrate.

The feature of the newly identified class of polymerases to selectively accept 2-amino-2′-deoxyadenosine 5′-triphosphate (dZTP) as a substrate and to reject deoxyadenosine 5′-triphosphate (dATP) as a substrate, i.e. to discriminate between these two substrates and to only incorporate dZTP, is surprising and an extremely striking feature. This seems to be the first example of a polymerase which excludes a dNTP from incorporation during polynucleotide chain synthesis. Even though it was, e.g., found previously that bacteriophage T4 DNA polymerase can synthesize DNA containing 5hmdC fully substituting fordC in vivo, this polymerase does not discriminate between these two bases in vitro. Moreover, all commercially available polymerases are capable of utilizing a wide variety of non-canonical dNTPs in vitro. The highly selective utilization of 2-amino-2′-deoxyadenosine 5′-triphosphate (dZTP) as a substrate instead of deoxyadenosine 5′-triphosphate (dATP) as a substrate shown for the novel family of DNA polymerases described herein opens up interesting possibilities for biotechnological applications, some of which will be described herein below.

The polymerases of the present invention will also be referred to herein as “PolZ” or “DpoZ” and the respective genes encoding such polymerases will be referred to as “dpoZ”.

As described in the appended Examples, the present inventors used a rather unusual way for identifying the new class of polymerases which are derived from bacteriophages belonging to the Siphoviridae family (and other, preferably closely related, bacteriophage families). The viruses of this cluster infect cellular hosts as distant as Gram-negative proteobacteria (Vibrio, Salmonella and Acinetobacter), Gram-positive actinobacteria (Arthrobacter and Gordonia) and cyanobacteria (Synechococcus) and dwell habitats as diverse as soil, freshwater and seawater. In short, the inventors first looked for genes which may show homology to the purZ gene of the phage S-2L by remote homology searching. In the following, they analyzed the genomes of the organisms which were identified to contain such tentative purZ homologues for sequences which might encode polymerases. In a further step, it was attempted to find out whether any of such genes which may encode a polymerase co-evolved with the identified purZ homologues. It was surprisingly found that it was possible to identify a group of tentative polymerase encoding genes (homologous to polA for DNA polymerase I) which occur in synteny with the purZ homologues. The biochemical and enzymological characterization of four of these polymerases which only show a low degree of sequence identity to each other and which are derived from four different phages which are only distantly related and which infect microbes as different as Vibrio, Gordonia, Acinetobacter and Arthobacter revealed that all encode polymerases which belong to a novel functional category of DNA polymerases that discriminate for 2-aminoadenine and against adenine in the dNTP substrate and prefer templates containing dZ over templates containing dA. The presumptive novel polymerase gene from Siphoviruses was designated dpoZ, in accordance with Demerec's nomenclature (Demerec et al., Genetics 54 (1966), 61-67; see also https://jb.as.org/content/nomenclature).

The sequences shown in SEQ ID NOs: 112 to 120 represent exemplary DpoZ polymerases identified in:
Acinetobacter phage SH-Ab15497 (SEQ ID NO: 113);
Salmonella phage PMBT28 (SEQ ID NO: 114);
Vibrio phage phiVC8 (SEQ ID NO: 112);
Alteromonas phage ZP6 (SEQ ID NO: 117);
Streptomyces phage Hiyaa (SEQ ID NO: 120);
Gordonia phage Ghobes (SEQ ID NO: 116);
Arthrobacter phage Wayne (SEQ ID NO: 115);
Microbacterium phage Goodman (SEQ ID NO: 118);
Microbacterium phage Theresita (SEQ ID NO: 119).

FIG. 6 shows an alignment of these sequences. Among each other, these sequences show sequence identities from 30% to 60%. Further corresponding polymerase encoding genes could be found in other genomes of phages, predominantly of Siphoviridae phages, by further remote homology searches and phylogenetic analysis as regards the presence of a PurZ homologue in the corresponding phage genome, preferably in close proximity to the identified polymerase gene. “Close proximity” in particular means that the identified polymerase gene can be found in a distance of less than 20 genes, preferably of less than 18 genes, even more preferably of less than 17 genes and most preferably of less than 16 genes away from the PurZ homologue. The amino acid sequences of the corresponding DpoZ polymerases are shown in SEQ ID NOs: 1 to 111 and 121 to 233. When comparing the amino acid sequences of all the sequences shown in SEQ ID NOs: 1 to 233 it is evident that the sequences show identities of as low as about 30%. Nevertheless, as is shown in the appended Examples, despite the low degree of sequence identity, it could be shown that the corresponding polymerases nevertheless have the same functionality of being able to discriminate between dZ and dA as a substrate and preferring templates which contain dZ over templates which contain dA. Thus, it is evident that the novel class of DNA polymerases showing the same functionality of being able to discriminate between dZ and dA as a substrate and preferring templates which contain dZ over templates which contain dA only show a low degree of sequence identity. They share as a feature that they can be identified in genomes of phages, in particular in genomes of such phages which also contain a gene encoding a PurZ homologue, preferably in close proximity to the gene encoding the polymerase. Such phages preferably belong to the family of Siphoviridae or closely related virus families.

Thus, the polymerase as described in the present application is an enzyme which shows the features (a) to (d) as listed above, wherein the sequence identity to any one of SEQ ID NOs: 1 to 233 listed in feature (a) is at least 50%, preferably at least 55%, more preferably at least 60%, even more preferably at least 65%, particularly preferred at least 70%, 75%, 80%, 85%, 90%, 95%, 96%, 97%, 98% or 99%.

A structural analysis of the identified novel type of DNA polymerase revealed that it structurally corresponds to the Klenow fragment of E. coli PolA lacking the 5′-exonuclease domain but retaining the 3′-exonuclease domain (see FIGS. 1 and 7). However, the overall sequence identity of the newly identified class of dpoZ polymerases to the E. coli Klenow fragment is less than 30%.

The sequences shown in SEQ ID NOs: 112 to 120 which are structurally more closely analyzed and partly functionally analyzed in the appended Examples are merely representative examples of polymerases characterized by the features defined above which could be identified in the genomes of different types of Siphoviridae phages and closely related phage families. Further sequences encoding such polymerases have been identified in a large number of phages, predominantly Siphiroviridae phages, using the above outlined approach and are listed in Table 1 and in SEQ ID NOs: 1 to 111 and 121 to 233.

Further polymerases which show the above features (a) to (d) can be identified by applying the following steps:

Searching the genomic sequence of a phage, preferably a Siphiroviridae phage, for a nucleotide sequence which is a homolog of the purZ gene of the phage S-2L by remote homology searching. If such a purZ homolog is found in the genome of the phage, searching the genome of said phage for a gene which codes for a polymerase, preferably a gene which codes for a polymerase which shows at least 25% sequence identity to any one of SEQ ID Nos: 1 to 233. The fact that the genome of the respective phage comprises a purZ homolog is an indication that a polymerase gene which can be identified in such a phage is a polymerase which fulfills the features (b) to (d) as set forth above. Whether this is indeed the case can be verified by assays known to the person skilled in the art and as described in the further below. Generally, it is found that the purZ homolog found in a phage genome, preferably a Siphiroviridae genome, is located in close proximity to the polymerase gene, i.e. not more than 20 genes away, more preferably not more than 18, 17 or 16 genes away.

As mentioned above, the polymerase described herein has an amino acid sequence which is at least 50% identical to the amino acid sequence shown in any one of SEQ ID NOs: 1 to 233; preferably the polymerase has an amino acid sequence which is at least 50% identical to the amino acid sequence shown in any one of SEQ ID NOs: 1 to 207, even more preferably it has an amino acid sequence which is at least 50% identical to the amino acid sequence shown in any one of SEQ ID NOs: 112 to 120, particularly preferred it has an amino acid sequence which is at least 50% identical to the amino acid sequence shown in any one of SEQ ID NOs: 113 to 116.

The term “polymerase” as used in the scope of the present invention refers to a DNA polymerase. A DNA polymerase is characterized by the ability to polymerize a polynucleotide chain comprising deoxynucleotides from deoxynucleoside-triphosphates (dNTPs) using as a template a DNA molecule.

The DpoZ polymerases of the present application are characterized in that they do not show 5′-exonuclease activity. Whether a polymerase shows 5′-exonuclease activity or not can be verified by the skilled person by methods known in the art. One possibility is to analyze the amino acid sequence of the polymerase as regards structural similarities to known polymerase domains which convey 5′-exonuclease activity. If no such domains can be found in the polymerase in question, this indicates that the respective polymerase does not show 5′-exonuclease activity. Moreover, 5′-exonuclease activity can be tested for by applying standard assays. Such a standard assay is, e.g. described in Setlow and Kornberg (J. Biol. Chem. 247 (1972), 232-240).

The DpoZ polymerases of the present application are furthermore characterized in that they accept 2-amino-2′-deoxyadenosine 5′-triphosphate (dZTP) as a substrate. This means that a DpoZ polymerase of the present invention is able to use dZTP as a substrate during DNA replication and can incorporate it into a growing polynucleotide chain. This activity can be tested in assays known to the person skilled in the art (see, e.g., (Wynne et al., PLoS ONE. 8, e70892 (2013)) and as described in the appended Examples. For example, this activity can be tested by using a short stretch (e.g. around 24 nucleotides) of polydT as a template and wherein the template comprises at its 5′-end a short sequence (e.g. around 18 nucleotides long) to which a primer sequence can be hybridized which provides for a 3′-end serving as a starting point for the polymerase for polymerization. Advantageously, the primer sequence comprises a marker/tag which allows the detection of any possible elongation products which may result from an elongation by a polymerase, such as a fluorescent label. Such a template with hybridized primer can then be incubated with dZTP and with the polymerase to be tested for its ability to use dZTP as a substrate. After incubation the products of the reaction are analyzed for a possible elongation of the primer sequence based on the template and the incorporation of dZTP into a polynucleotide, e.g. by gel electrophoresis and detection of the marker/tag carried by the primer sequence.

In a preferred embodiment, the ability of a polymerase of the present invention to use dZTP as a substrate is tested according to a primer extension assay as described in the Materials and Methods section of the appended Examples. More specifically, such an assay is preferably designed as follows:

A reaction mix is prepared consisting of 100 μL volume containing 1 μM of the FAM-fluorescent-labeled 20-mer primer X1903 (shown in FIG. 3A (1) or (2); see Table 3 for the primer sequence), 3 μM of the 42-mer template comprising polydT (FIG. 3A (1) or (2)), 20 mM Tris-HCl (pH 7,5) buffer, 200 mM NaCl (or alternatively 50 mM NaCl), 1 mM DTT, 5 mM MgCl₂ and 1 mM dZTP. Primer:template duplexes are annealed by denaturation 2 min at 95° C., and cooled down to 4° C. (0.1° C./s). The polymerisation assay is started by adding 0.034 μM of DNA polymerase after 5 min of primer-template duplexes pre-heating at 37° C. Reactions are incubated at 37° C. for 30 min and 10 μL of reaction is mixed to 10 μL of quenching buffer (98% formamide, 10 mM EDTA). Extension products are separated by electrophoresis migration in denaturing polyacrylamide gels (7M, 1×TBE, 17% acrylamide gel). Gels are analyzed using the G-BOX (Ozyme) and band intensities are quantified with Genetools software (Syngene).

As a positive control, a polymerase which is known to accept dZTP as a substrate (e.g. any of the DpoZ polymerases as described in the present application or E. coli Klenow fragment) can be employed in a parallel assay. In order to assess in the gel electrophoresis analysis of the extension products whether an extension of the primer due to an incorporation of dZTP has occurred, the primer sequence and the expected full-length product are also analysed in parallel on the gel.

The polymerase is classified as being able to use dZTP as a substrate and to incorporate it into a growing polynucleotide chain if such an assay leads to a full-length extension product of the used template.

Such an assay can also be designed so as to quantify the percentage of primer molecules which has been extended to a full-length product. A preferable assay for carrying out the polymerisation experiments using different substrates (or templates) and subsequent quantification is an assay as described in Wynne et al. (PLOS ONE, 8 (2013), e70892; doi:10.1371/journal.pone.0070892). For example, such a quantification of the band intensities obtained in the gel can be done by using the Genetools software (Syngene).

If such a quantification is done, a polymerase is classified as being able to use dZTP as a substrate if at least 20%, more preferably at least 50%, even more at least 70% of the primer molecules are extended to a full-length product.

The DpoZ polymerases of the present application are furthermore characterized in that they do not accept deoxyadenosine 5′-triphosphate (dATP) as a substrate. In the context of the present invention the phrase “do not accept deoxyadenosine 5′-triphosphate (dATP) as a substrate” means that a dpoZ polymerase of the present invention is not able to use dATP as a substrate during DNA replication and cannot incorporate it into a growing polynucleotide chain or can only accept dATP as a substrate and incorporate it to a very low degree. This activity can be tested in assays known to the person skilled in the art (see, e.g., (Wynne et al., PLoS ONE. 8, e70892 (2013)) and as described in the appended Examples. For example, this activity can be tested by using a short stretch (e.g. around 24 nucleotides) of polydT as a template and wherein the template comprises at its 5′-end a short sequence (e.g. around 18 nucleotides long) to which a primer sequence can be hybridized which provides for a 3′-end serving as a starting point for the polymerase for polymerization. Advantageously, the primer sequence comprises a marker/tag which allows the detection of any possible elongation products which may result from an elongation by a polymerase, such as a fluorescent label. Such a template with hybridized primer can then be incubated with dATP and with the polymerase to be tested for its ability to use dATP as a substrate. After incubation the products of the reaction are analyzed for a possible elongation of the primer sequence based on the template and the incorporation of dATP into a polynucleotide, e.g. by gel electrophoresis and detection of the marker/tag carried by the primer sequence.

In a preferred embodiment, the ability of a polymerase of the present invention to use dATP as a substrate or the lack of such an ability is tested according to a primer extension assay as described in the Materials and Methods section of the appended Examples. More specifically, such an assay is preferably designed as follows:

A reaction mix is prepared consisting of 100 μL volume containing 1 μM of the FAM-fluorescent-labeled 20-mer primer X1903 (shown in FIG. 3A (1) or (2); for the primer sequence see Table 3), 3 μM of the 42-mer template comprising polydT (FIG. 3A (1) or (2)), 20 mM Tris-HCl (pH 7,5) buffer, 200 mM NaCl (or alternatively 50 mM NaCl), 1 mM DTT, 5 mM MgCl₂ and 1 mM dATP. Primer:template duplexes are annealed by denaturation 2 min at 95° C., and cooled down to 4° C. (0.1° C./s). The polymerisation assay is started by adding 0.034 μM of DNA polymerase after 5 min of primer-template duplexes pre-heating at 37° C. Reactions are incubated at 37° C. for 30 min and 10 μL of reaction is mixed to 10 μL of quenching buffer (98% formamide, 10 mM EDTA). Extension products are separated by electrophoresis migration in denaturing polyacrylamide gels (7M, 1×TBE, 17% acrylamide gel). Gels are analyzed using the G-BOX (Ozyme) and band intensities are quantified with Genetools software (Syngene).

As a positive control, a polymerase which is known to accept dATP as a substrate (e.g. E. coli Klenow fragment) can be employed in a parallel assay. In order to assess in the gel electrophoresis analysis of the extension products whether an extension of the primer due to an incorporation of dATP has occurred, the primer sequence and the expected full-length product are also analysed in parallel on the gel.

The polymerase is classified as not being able to use dATP as a substrate and to incorporate it into a growing polynucleotide chain if such an assay does not lead to a full-length extension product of the used template.

The feature that the DpoZ polymerases as described herein can accept dZTP as a substrate but cannot accept dATP as a substrate also means that, in a situation in which such a polymerase is used for the synthesis of a DNA strand based on a template DNA in the presence of both, dZTP and dATP, the polymerase selectively incorporates dZ into the produced polynucleotide chain but does not incorporate dA. Since a minor degree of acceptance of dATP by a DpoZ polymerase cannot completely be excluded, the term “selectively incorporates” means that the polymerase incorporates at more than 95% of dT residues in the template strand a dZ instead of a dA in the newly synthesized strand, even more preferably at more than 98% of dT residues in the template strand.

In a preferred embodiment, the DpoZ polymerases of the present invention are furthermore characterized in that they do not accept deoxyinosine 5′-triphosphate (dITP) as a substrate. In the context of the present invention the phrase “do not accept deoxyinosine 5′-triphosphate (dITP) as a substrate” means that a dpoZ polymerase of the present invention is not able to use dITP as a substrate during DNA replication and cannot incorporate it into a growing polynucleotide chain or can only accept dITP as a substrate and incorporate it to a very low degree. This activity can be tested in assays known to the person skilled in the art (see, e.g., (Wynne et al., PLoS ONE. 8, e70892 (2013)) and as described in the appended Examples. For example, this activity can be tested by using a short stretch (e.g. around 24 nucleotides) of polydC as a template and wherein the template comprises at its 5′-end a short sequence (e.g. around 18 nucleotides long) to which a primer sequence can be hybridized which provides for a 3′-end serving as a starting point for the polymerase for polymerization. Advantageously, the primer sequence comprises a marker/tag which allows the detection of any possible elongation products which may result from an elongation by a polymerase, such as a fluorescent label. Such a template with hybridized primer can then be incubated with dITP and with the polymerase to be tested for its ability to use dITP as a substrate. After incubation the products of the reaction are analyzed for a possible elongation of the primer sequence based on the template and the incorporation of dITP into a polynucleotide, e.g. by gel electrophoresis and detection of the marker/tag carried by the primer sequence.

In a preferred embodiment, the ability of a polymerase of the present invention to use dITP as a substrate or the lack of such an ability is tested according to a primer extension assay as described in the Materials and Methods section of the appended Examples. More specifically, such an assay is preferably designed as follows:

A reaction mix is prepared consisting of 100 μL volume containing 1 μM of the FAM-fluorescent-labeled 20-mer primer X1903 (shown in FIG. 8; for the primer sequence see Table 3), 3 μM of the 42-mer template comprising polydC (X1929; for the sequence see Table 3; FIG. 8), 20 mM Tris-HCl (pH 7,5) buffer, 200 mM NaCl (or alternatively 50 mM NaCl), 1 mM DTT, 5 mM MgCl₂ and 1 mM dITP. Primer:template duplexes are annealed by denaturation 2 min at 95° C., and cooled down to 4° C. (0.1° C./s). The polymerisation assay is started by adding 0.034 μM of DNA polymerase after 5 min of primer-template duplexes pre-heating at 37° C. Reactions are incubated at 37° C. for 30 min and 10 μL of reaction is mixed to 10 μL of quenching buffer (98% formamide, 10 mM EDTA). Extension products are separated by electrophoresis migration in denaturing polyacrylamide gels (7M, 1×TBE, 17% acrylamide gel). Gels are analyzed using the G-BOX (Ozyme) and band intensities are quantified with Genetools software (Syngene).

As a positive control, a polymerase which is known to accept dITP as a substrate (e.g. E. coli Klenow fragment) can be employed in a parallel assay. In order to assess in the gel electrophoresis analysis of the extension products whether an extension of the primer due to an incorporation of dITP has occurred, the primer sequence and the expected full-length product are also analysed in parallel on the gel.

The polymerase is classified as not being able to use dITP as a substrate and to incorporate it into a growing polynucleotide chain if such an assay does not lead to a full-length extension product of the used template.

The DpoZ polymerases of the present invention are characterized in that they accept dCTP, dGTP and dTTP as substrates. The ability to use any of these nucleotides as substrates can be assessed by primer extension assays as described above in which, however, a corresponding template (polydG for dCTP; polydC for dGTP and polydA for dTTP) is used and the corresponding nucleotide is added to the reaction mix.

In one embodiment of the present invention, the DpoZ polymerases of the present invention are furthermore characterized in that they show 3′-exonuclease activity. Whether a polymerase shows 3′-exonuclease activity or not can be verified by the skilled person by methods known in the art. One possibility is to analyze the amino acid sequence of the polymerase as regards structural similarities to known polymerase domains which convey 3′-exonuclease activity. If no such domains can be found in the polymerase in question, this indicates that the respective polymerase does not show 3′-exonuclease activity.

The DpoZ polymerases of the present invention are in particular characterized in that they show 3′-exonuclease activity on DNA single-strands. In a preferred embodiment, the DpoZ polymerases of the present application are furthermore characterized in that they show 3′-exonuclease activity on DNA double-strands. Whether a polymerase shows 3′-exonuclease activity on DNA single-strands or not or whether a polymerase shows 3′-exonuclease activity on DNA double-strands or not can be verified by the skilled person by methods known in the art. For example, 3′-exonuclease activity on DNA single-strands or DNA double-strands can be tested for by applying standard assays (see e.g. Derbyshire et al., EMBO J. 10 (1991), 17-24).

An example for an assay for assessing 3′-exonuclease activity is described in the Example section and the corresponding results are shown in FIGS. 4A and B. Thus, an assay for analyzing whether a given protein has 3′-exonuclease activity on a DNA single-strand can, e.g., be designed in such a manner that a 5′-labeled (e.g. fluorescent labeled) DNA single-strand is incubated (in the absence of dNTPS) with the respective polymerase to be tested for different time intervals (e.g. 5 minutes and 60 minutes) at an appropriate temperature (e.g. 37° C.). The used DNA single-strand preferably has the sequence as shown in FIG. 4A. The reaction products are then loaded onto a denaturing 17% polyacrylamide gel and separated by gel electrophoresis. As a positive control a reaction can be used employing a polymerase which is known to show 3′-exonuclease activity on DNA single-strands, such as the E. coli Klenow fragment. On the gel, a corresponding amount of the labeled untreated DNA single-strand is used as a control. If the gel analysis shows that after 60 minutes of incubation the amount of single-stranded DNA is reduced compared to the untreated single-stranded DNA (with a reduction meaning a reduction of at least 10%, preferably of at least 50%, even more preferably of at least 90%) this indicates 3′-exonuclease activity on DNA single-strands.

An assay for analyzing whether a given protein has 3′-exonuclease activity on a DNA double-strand can, e.g., be designed in such a manner that a 5′-labeled (e.g. fluorescent labeled) primer is annealed to an unlabeled template so as to create a partly double-stranded DNA molecule and the resulting duplex is incubated (in the absence of dNTPS) with the respective polymerase to be tested for different time intervals (e.g. 5 minutes and 60 minutes) at an appropriate temperature (e.g. 37° C.). The used primer and template strand preferably have the sequences as shown in FIG. 4B. The reaction products are then loaded onto a denaturing 17% polyacrylamide gel and separated by gel electrophoresis. As a positive control a reaction can be used employing a polymerase which is known to show 3′-exonuclease activity on DNA double-strands. On the gel, a corresponding amount of the untreated labeled primer is used as a control. In case the polymerase shows no 3′-exonuclease activity on the annealed duplex, a band corresponding in length and strength to the untreated labeled primer is seen. In case the polymerase shows 3′-exonuclease activity on the annealed duplex, the band corresponding in length to the untreated labeled primer shows a reduced intensity and, preferably, distinct bands with shorter length can be observed. If the gel analysis shows that after 60 minutes of incubation the amount of the signal corresponding to the untreated labeled primer is reduced compared to the untreated labeled primer (with a reduction meaning a reduction of at least 20%, preferably of at least 30%, even more preferably of at least 50%) this indicates 3′-exonuclease activity on DNA double-strands. Moreover, the occurrence of bands with a shorter length than the untreated labeled primer also is an indication for 3′-exonuclease activity on DNA double-strands.

In another embodiment of the present invention, the DpoZ polymerases of the present application are furthermore characterized in that they do not show 3′-exonuclease activity.

In this embodiment the DpoZ polymerases of the present invention are in particular characterized in that they do not show 3′-exonuclease activity on DNA single-strands. In a further preferred embodiment, the DpoZ polymerases of the present application are furthermore characterized in that they do not show 3′-exonuclease activity on DNA double-strands. Whether a polymerase shows 3′-exonuclease activity on DNA single-strands or not or whether a polymerase shows 3′-exonuclease activity on DNA double-strands or not can be verified by the skilled person by methods known in the art and as described above.

Thus, an example for an assay for assessing 3′-exonuclease activity is described in the Example section and the corresponding results are shown in FIGS. 4A and B. Thus, an assay for analyzing whether a given protein has 3′-exonuclease activity on a DNA single-strand or not can, e.g., be designed in such a manner that a 5′-labeled (e.g. fluorescent labeled) DNA single-strand is incubated (in the absence of dNTPS) with the respective polymerase to be tested for different time intervals (e.g. 5 minutes and 60 minutes) at an appropriate temperature (e.g. 37° C.). The used DNA single-strand preferably has the sequence as shown in FIG. 4A. The reaction products are then loaded onto a denaturing 17% polyacrylamide gel and separated by gel electrophoresis. As a positive control a reaction can be used employing a polymerase which is known to show 3′-exonuclease activity on DNA single-strands, such as the E. coli Klenow fragment. On the gel, a corresponding amount of the labeled untreated DNA single-strand is used as a control. If the gel analysis shows that after 60 minutes of incubation the amount of single-stranded DNA is not reduced compared to the untreated single-stranded DNA (“not reduced” meaning in this context a reduction of less than 10%, preferably of less than 8%, even more preferably of at least 5%) this indicates that the respective polymerase does not have 3′-exonuclease activity on DNA single-strands.

An assay for analyzing whether a given protein has 3′-exonuclease activity on a DNA double-strand or not can, e.g., be designed in such a manner that a 5′-labeled (e.g. fluorescent labeled) primer is annealed to an unlabeled template so as to create a partly double-stranded DNA molecule and the resulting duplex is incubated (in the absence of dNTPS) with the respective polymerase to be tested for different time intervals (e.g. 5 minutes and 60 minutes) at an appropriate temperature (e.g. 37° C.). The used primer and template strand preferably have the sequences as shown in FIG. 4B. The reaction products are then loaded onto a denaturing 17% polyacrylamide gel and separated by gel electrophoresis. As a positive control a reaction can be used employing a polymerase which is known to show 3′-exonuclease activity on DNA double-strands. On the gel, a corresponding amount of the untreated labeled primer is used as a control. In case the polymerase shows no or a reduced 3′-exonuclease activity on the annealed duplex, a band corresponding in length and strength to the untreated labeled primer is seen. If the gel analysis shows that after 60 minutes of incubation the amount of the signal corresponding to the untreated labeled primer is not reduced compared to the untreated labeled primer (“not reduced” meaning in this context a reduction of less than 10%, preferably of less than 8%, even more preferably of at least 5%) this indicates that there is no 3′-exonuclease activity on DNA double-strands.

As is illustrated in the appended Examples, the naturally occurring DpoZ polymerases of the present invention show 3′-exonuclease activity. However, for practical applications, i.e. the mass production of nucleic acids, such as plasmids, it may be advantageous to provide variants of such polymerases which do no longer show 3′-exonuclease activity. As shown in the appended Examples, the polymerases of the present invention are structurally related to the Klenow fragment of the E. coli polymerase and, accordingly, also show a domain which is structurally related to the domain of the Klenow fragment which conveys the 3′-exonuclease activity. As also shown in the appended Examples, it is possible to prepare variants of a naturally DpoZ polymerase (which can be identified in phage genomes) which do no longer show 3′-exonuclease activity. This can be achieved by effecting in the amino acid sequence of the DpoZ polymerase a substitution at a position which corresponds to position 424 of E. coli DNA pol 1. As shown in FIG. 1, the amino acid sequences of DpoZ polymerases can easily be aligned with the amino acid sequence of E. coli pol I (encoded by the polA gene). The amino acid “D” at position 424 of the amino acid sequence of the E. coli pol I enzyme lies within the domain which is responsible for 3′-exonuclease activity of the E. coli enzyme and is strongly conserved among the DpoZ enzymes analyzed so far (see FIG. 7). A substitution of “D” to “A” in either the E. coli Klenow fragment or in any of the DpoZ polymerases analyzed in the appended Examples leads to a loss of 3′-exonuclease activity.

Thus, in one preferred embodiment the DpoZ polymerase according to the present invention is a polymerase as defined above which does not have 3′-exonuclease activity and which is characterized in that it contains in its amino acid sequence a substitution in comparison to the wildtype sequence from which it is derived at the position which corresponds to position D424 of the E. coli pol 1. In a preferred embodiment, the substitution is a substitution by an alanine (A). In other preferred embodiments the DpoZ polymerase according to the present invention is a polymerase as defined above which does not have 3′-exonuclease activity and which is characterized in that it contains in its amino acid sequence a substitution in comparison to the wildtype sequence from which it is derived at the position which corresponds to position D355 or D501 of the E. coli pol 1. In a preferred embodiment, the substitution is a substitution by an alanine (A). It is known, e.g. from Derbyshire et al. (EMBO J. 10 (1991), 17-24) that these positions (as position D424) are essential for 3′-exonuclease activity.

The present invention also relates to a DpoZ polymerase encoded by a nucleic acid molecule of the present invention, wherein said DpoZ polymerase does not have 3′-exonuclease activity, preferably by a DpoZ polymerase which shows a substitution as described above.

A polymerase of the present invention is characterized in that it is able to use as a template a DNA molecule which contains dZ nucleotides instead of dA nucleotides. This ability can be tested in a primer extension assay as described above in which, however, a template is used which contains only dZ nucleotides or a template which comprises a random sequence and which contains dZ nucleotides instead of dA nucleotides. The occurrence of a full-length elongation product in such a primer-extension assay is indicative of the ability of the polymerase to use a template which comprises dZ instead of dA for DNA synthesis. A corresponding assay is also described in the Example section and is illustrated in FIGS. 3 and 5.

In one preferred embodiment, a polymerase of the present invention is characterized in that it is able to use as a template a DNA molecule which contains dZ nucleotides instead of dA nucleotides and it is also able to use as a template a DNA molecule which contains dA nucleotides. The ability of a polymerase to use as a template a DNA molecule which contains dA nucleotides can be tested in a primer extension assay as described above in which, however, a template is used which contains only dA nucleotides or a template which comprises a random sequence and which contains dA nucleotides (but no dZ nucleotides). The occurrence of a full-length elongation product in such a primer-extension assay is indicative of the ability of the polymerase to use a template which comprises A for DNA synthesis. A corresponding assay is described in the Example section and is illustrated in FIGS. 3 and 5. It is shown in the Examples (see FIGS. 3 and 5) that, e.g., the DpoZ polymerase of Vibrio phage phiVC8 (SEQ ID NO: 112) and that of Acinetobacter phage SH-Ab15497 (SEQ ID NO: 113) can use a template containing either Z or A deoxynucleotides.

In another preferred embodiment, a polymerase of the present invention is characterized in that it is able to use as a template a DNA molecule which contains dZ nucleotides instead of dA nucleotides, but it is not able to use as a template a DNA molecule which contains dA nucleotides. The ability of a polymerase to use as a template a DNA molecule which contains dA nucleotides can be tested in a primer extension assay as described above in which, however, a template is used which contains only dA nucleotides or a template which comprises a random sequence and which contains dA nucleotides (but no dZ nucleotides). If no full-length elongation products can be detected in such a primer-extension assay, this is indicative of the inability of the polymerase to use a template which comprises dA for DNA synthesis. A corresponding assay is described in the Example section and is illustrated in FIGS. 3 and 5.

It is shown in the Examples (see FIGS. 3 and 5) that, e.g., the DpoZ polymerase of Gordonia phage Ghobes (SEQ ID NO: 117) and that of Arthrobacter phage Wayne (SEQ ID NO: 116) can use a template containing dZ nucleotides but cannot use a template containing dA nucleotides.

As described above and as evident from FIGS. 1 and 7, the DpoZ polymerases of the present invention are structurally related to the E. coli pol I enzyme. However, there are also characteristic structural differences to the E. coli pol I enzyme. For example, a DpoZ polymerase as described herein is preferably characterized in that it does not show in its amino acid sequence a tyrosine residue at a position which corresponds to position Y766 of the E. coli pol I enzyme (SEQ ID NO: 234) but shows a different amino acid at this position, preferably a phenylalanine residue.

The term “recombinant nucleic acid molecule which comprises a nucleotide sequence encoding a polymerase” refers to a nucleic acid molecule which comprises a nucleotide sequence encoding a polymerase as described above and at least one nucleotide sequence which does not naturally occur in direct physical connection with such a nucleotide sequence encoding said polymerase. Thus, the term “recombinant nucleic acid molecule which comprises a nucleotide sequence encoding a polymerase” excludes a naturally occurring phage genome comprising a nucleotide sequence encoding a polymerase as defined above. In a preferred embodiment, the recombinant nucleic acid molecule comprises a nucleotide sequence encoding a polymerase as described above which is operatively linked to a heterologous promoter which allows for its expression in prokaryotic or eukaryotic cells. The term “heterologous” means that the promoter which is operatively linked to the nucleotide sequence which encodes the polymerase is different from the natural promoter which naturally is located in front of the sequence encoding the polymerase and which drives the expression of the gene in the phage genome.

The term “operatively linked” or “operably linked” as used herein refers to a linkage between one or more expression control sequences, like a promoter, and the coding region in the polynucleotide to be expressed in such a way that expression is achieved under conditions compatible with the expression control sequence. Expression comprises transcription of the respective nucleotide sequence, preferably into a translatable mRNA. Regulatory elements ensuring expression in as in prokaryotes, such as bacteria, and eukaryotes, such as fungal, animal and plant cells, are well known to those skilled in the art. They encompass promoters, enhancers, termination signals, targeting signals and the like. Examples are given further below in connection with explanations concerning vectors.

Promoters for use in connection with the nucleic acid molecule may be homologous or heterologous with regard to the gene to be expressed and/or the cell in which they are intended to be employed. Suitable promoters are for instance promoters which lend themselves to constitutive expression. However, promoters which are only activated at a point in time determined by external influences can also be used. Artificial and/or chemically inducible promoters may be used in this context.

The recombinant nucleic acid molecule can further comprise expression control sequences operably linked to said nucleotide sequence encoding a polymerase. These expression control sequences may be suited to ensure transcription and synthesis of a translatable RNA in bacteria or fungi.

In addition, it is possible to insert different mutations into the polynucleotides by methods usual in molecular biology (see for instance Sambrook and Russell (2001), Molecular Cloning: A Laboratory Manual, CSH Press, Cold Spring Harbor, NY, USA), leading to the synthesis of polypeptides possibly having modified biological properties. The introduction of point mutations is conceivable at positions at which a modification of the amino acid sequence for instance influences the biological activity or the regulation of the polypeptide.

It is, e.g., described in the Example section how mutants of the described polymerases can be produced which show no or a strongly reduced 3′-exonuclease activity.

Moreover, mutants possessing a modified substrate or product specificity can be prepared.

Preferably, such mutants show an increased activity. Alternatively, mutants can be prepared the catalytic activity of which is abolished without losing substrate binding activity.

Furthermore, the introduction of mutations into the polynucleotides encoding an polymerase as defined above allows the gene expression rate and/or the activity of the enzymes encoded by said polynucleotides to be reduced or increased.

For genetically modifying bacteria or fungi, the polynucleotides encoding an enzyme as defined above or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, NY, USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods. The polynucleotide encoding the polymerase can be expressed so as to lead to the production of a polypeptide having the activities described above. An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

The present invention also relates to a vector, such as a plasmid, comprising the recombinant nucleic acid molecule as described above. One example of a vector is construct which allows for the introduction into a desired host cell and the presence of the recombinant nucleic acid molecule in the host cell either in extrachromosomal form (e.g., in the form of a replicating plasmid) or integrated into the host cell's genome. Such a construct may also be designed as an expression vector which contains regulatory elements which allow for the expression of the nucleotide sequence encoding the polymerase in the desired host cell. Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

The present invention also relates to a recombinant host cell comprising a recombinant nucleic acid molecule of the present invention or being transformed with a nucleotide sequence encoding a polymerase as defined above. The term “recombinant” in this context means that the host cell has been genetically modified by the introduction of the recombinant nucleic acid molecule of the present invention or by a nucleotide sequence encoding a polymerase as defined above. Thus, the recombinant host cell is characterized by the fact that it naturally does not contain a nucleotide sequence encoding a polymerase as defined herein above. In a preferred embodiment, the nucleotide sequence encoding a polymerase which is used to transform the host cell or which is contained in the recombinant nucleic acid molecule which is introduced into the host cell is recombinant in the sense that comprises a nucleotide sequence encoding a polymerase as described above which is operatively linked to a heterologous promoter which allows for its expression in said host cell. The term “heterologous” means that the promoter which is operatively linked to the nucleotide sequence which encodes the polymerase is different from the natural promoter which is located in front of the sequence encoding the polymerase and naturally drives the expression of the gene in the phage genome.

The host cell may be any possible cell, such as a microorganism or an animal or plant cell. The term “microorganism” in the context of the present invention refers to bacteria, as well as to fungi, such as yeasts, and also to algae and archaea. In one preferred embodiment, the microorganism is a bacterium. In principle any bacterium can be used as a host cell. Preferred bacteria are bacteria of the genus Bacillus, Clostridium, Corynebacterium, Pseudomonas, Zymomonas or Escherichia. In a particularly preferred embodiment the bacterium belongs to the genus Escherichia and even more preferred to the species Escherichia coli. In another preferred embodiment the bacterium belongs to the species Pseudomonas putida or to the species Zymomonas mobilis or to the species Corynebacterium glutamicum or to the species Bacillus subtilis.

The host cell may also be an extremophilic bacterium such as Thermus thermophilus, or anaerobic bacteria from the family Clostridiae.

In another preferred embodiment the microorganism is a fungus, more preferably a fungus of the genus Saccharomyces, Schizosaccharomyces, Aspergillus, Trichoderma, Kluyveromyces or Pichia and even more preferably of the species Saccharomyces cerevisiae, Schizosaccharomyces pombe, Aspergillus niger, Trichoderma reesei, Kluyveromyces marxianus, Kluyveromyces lactis, Pichia pastoris, Pichia torula or Pichia utilis.

In another embodiment, the host cell is a photosynthetic microorganism expressing at least one enzyme for the conversion according to the invention as described above. Preferably, the microorganism is a photosynthetic bacterium, or a microalgae. In a further embodiment the microorganism is an algae, more preferably an algae belonging to the diatomeae.

In one preferred embodiment the host cell is a host cell which is able to synthesize dZTP. If a cell is not naturally able to synthesize dZTP this ability can be conferred to the cell by providing it with nucleic acid molecules which encode enzymes which confer this ability. As described above, the phage S-2L displays in its genome a gene, designated purZ, coding for a protein distantly related to succinoadenylate synthase (EC 6.3.4.4) the cellular enzyme of adenine biosynthesis, encoded by purA. It has been found that the protein encoded by this purZ gene is able to react aspartate with deoxyguanylate into dSMP (N6-succino-2-amino-2′-deoxyadenylate). Since these substrates naturally occur in basically every cellular organism, a host cell can be equipped with the ability to synthesize dSMP by introducing into such a host cell a nucleic acid molecule which encodes a PurZ protein and expressing it. Nucleic acid sequences which encode a purZ protein are known from the genome of the phage (i.e. the nucleotide sequence which encodes a protein which is related to PurA.

The dSMP which is synthesized by the PurZ protein can then be further converted into dZTP by enzymes which usually occur in cellular organisms. For example, the dSMP can be further converted in to dZMP by lyase, such as an adenylosuccinate lyase (EC 4.3.2.2), which is encoded by the purB gene. Corresponding enzymes and encoding nucleic acid molecules are known from various organisms, such as E. coli or bacteria of the genus Vibrio, e.g. Vibrio cholerae. The thus produced dZMP can then be further converted into dZTP by kinases, e.g. by a guanylate kinase (EC 2.7.4.8; encoded by the gmk gene) and a nucleoside diphosphate kinase (EC 2.7.4.6; encoded by the ndk gene).

Thus, the present invention also relates to a host cell which expresses a DpoZ polymerase as described herein above and which is furthermore capable of converting dZ into dZTP, preferably due to the expression of an adenylosuccinate lyase (EC 4.3.2.2), a guanylate kinase (EC 2.7.4.8) and a nucleoside diphosphate kinase (EC 2.7.4.6).

In another preferred embodiment the host cell is capable of importing dZTP from the exterior into the cell. This can, e.g., be achieved by genetically modifying the host cell so as to express a dNTP transporter as, e.g. described in Pezo et al. (ACS Synth. Biol.; DOI: 10.1021/acssynbio.8b00048).

The transformation of the host cell with a recombinant nucleic acid molecule or a vector as described above can be carried out by standard methods, as for instance described in Sambrook and Russell (2001), Molecular Cloning: A Laboratory Manual, CSH Press, Cold Spring Harbor, NY, USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

Furthermore, the present invention relates to a method for the production of a DNA molecule comprising 2-amino-2′-deoxyadenosine (dZ) instead of deoxyadenosine (dA) comprising the steps of:
(i) Providing a template DNA molecule to be replicated;
(ii) Providing dNTPs and wherein said dNTPs include 2-amino-2′-deoxyadenosine-triphosphate (dZTP);
(iii) Providing a polymerase as described herein above; and
(iv) Incubating the components of (i) to (iii).

As described above, the DpoZ polymerases described herein allow to produce DNA molecules which comprise 2-amino-2′-deoxyadenosine (dZ) instead of deoxyadenosine (dA). Since the described DpoZ polymerases do not accept dATP as a substrate, DNA molecules which are produced by a DpoZ polymerase as described in the present invention comprise dZ nucleotides instead of dA nucleotides. Thus, even in the presence of dATP and dZTP at the same time, the DpoZ polymerases will selectively incorporate dZ into a growing nucleotide chain instead of dA.

The method for the production of a DNA molecule may be carried out in vitro or in vivo.

For carrying out the method in vitro, the different components mentioned in (i) to (iii) are provided in a reaction vessel (e.g. an Eppendorf tube or the well of a 96-well plate) in a suitable reaction medium (appropriate buffer etc., e.g. as described in the Example section).

The template DNA molecule to be replicated can be any suitable DNA molecule. Such a DNA molecule is provided in a form which allows for the polymerase to act on it. For example, the template can be provided in the form of a single stranded DNA molecule to which a primer molecule is annealed which provides for a 3′-end to which the polymerase can attach nucleotides thereby synthesizing a polynucleotide. In principle, the template can be furnished in any suitable form which provides for a template strand to be replicated and a partly double-stranded region with a free 3′-end as a starting point for DNA synthesis for the polymerase. Suitable forms are known to the person skilled in the art.

The dNTPs provided as component (ii) include dZTP. The dNTPs also include other dNTPs which are necessary for the synthesis of the DNA molecule. Typically, these are the naturally occurring dNTPs dTTP, dCTP and dGTP. However, these may be substituted by modified versions of these dNTPs in as far as the DpoZ polymerase employed in the method is able to accept such modified dNTPs as substrates and to incorporate them into a polynucleotide chain. Moreover, it is also possible to include in such a reaction dATP in case it is intended to benefit in such a method from the ability of the polymerase to selectively incorporate into the produced DNA molecule dZ instead of dA, e.g. for selection purposes or the like.

For an in vitro method, the polymerase in component (iii) can be provided in any suitable form to the reaction mixture. Preferably, the polymerase is provided as a purified enzyme.

In case of an in vitro method, the incubation of step (iv) of the method according to the present invention is carried out under appropriate conditions which allow for the polymerase to be active. Corresponding conditions can be determined by the skilled person by routine experiments. Exemplary conditions are described in the appended Examples and can be varied or optimized for a particular polymerase by the skilled person according to routine measures.

The method according to the present invention for the production of a DNA molecule comprising 2-amino-2′-deoxyadenosine (dZ) instead of deoxyadenosine (dA) can also be carried out in vivo. For this, the provision of the DNA template can be achieved by making sure that the DNA molecule to be replicated is in the cell used for the method and can be accessed by the polymerase.

If the method is carried out in vivo, the dZTP may be provided by the cells themselves or it may be provided externally. Thus, in the former case, the step of providing 2-amino-2′-deoxyadenosine-triphosphate (dZTP) is achieved by having dZTP synthesized by the cells. This may be accomplished by providing dZ in the culture medium and by making sure that the cells which are used in the method have the ability to convert dZ into dZTP. How this can be achieved is described above. In the latter case, i.e. the provision of dZTP externally, the dZTP can be imported into the cells with the help of a dNTP transporter (see, e.g., Pezo et al. (ACS Synth. Biol.; DOI: 10.1021/acssynbio.8b00048).

The remaining dNTPs are normally provided by the cells themselves. Typically, these are the naturally occurring dNTPs dTTP, dATP, dCTP and dGTP. However, these may be substituted by modified versions of these dNTPs in as far as the DpoZ polymerase employed in the method is able to accept such modified dNTPs as substrates and to incorporate them into a polynucleotide chain. Such modified dNTPs can either be synthesized by the cells themselves or they can be provided externally and can be imported into the cells, e.g. with the help of a dNTP transporter (see, e.g., Pezo et al. (ACS Synth. Biol.; DOI: 10.1021/acssynbio.8b00048). When the method according to the present invention is carried out in vitro, the provision of the polymerase in step (iii) is normally achieved by having the polymerase expressed by the cells which are employed in the method. Such cells are normally host cells according to the present invention into which a nucleotide sequence encoding the corresponding polymerase has been introduced and which are therefore genetically modified. The nucleotide sequence encoding the polymerase is generally linked to an expression control region (including a promoter, preferably a heterologous promoter) which allows for expression of the polymerase in the cells.

If the method is carried out in vivo, the incubation step (iv) includes the culturing of the cells which are used for the method. Such culturing could, e.g., be a small-scale culturing on a laboratory scale in corresponding flasks or on culture plates, or it can be large-scale fermentation in a bioreactor.

In one embodiment of the method of the present invention, preferably in a case in which the method is carried out in vitro, the template DNA molecule comprises dZ nucleotides instead of dA nucleotides.

The method of method of the present invention for the production of a DNA molecule comprising 2-amino-2′-deoxyadenosine (dZ) instead of deoxyadenosine (dA) may furthermore comprise the step of recovering the produced DNA molecule comprising 2-amino-2′-deoxyadenosine (dZ).

The present invention also relates to the use of a DpoZ polymerase as described herein-above for the production of a DNA molecule comprising 2-amino-2′-deoxyadenosine (dZ) instead of deoxyadenosine (dA). In particular, the present invention relates to such a use in which the production of the DNA molecule is performed in the presence of dZTP and dATP.

Moreover, the present invention relates to a composition comprising a recombinant nucleic acid molecule encoding a DpoZ polymerase as described herein-above, a vector as described above or a host cell as described above and dZTP (and optionally further dNTPS like dTTP, dGTP and dCTP) as well as to a composition comprising a DpoZ polymerase as described herein-above and dZTP (and optionally further dNTPS like dTTP, dGTP and dCTP). Such a composition may optionally also comprise dATP.

The present invention also relates to a kit comprising (in separate compartments) a recombinant nucleic acid molecule encoding a DpoZ polymerase as described herein-above, a vector as described above or a host cell as described above and dZTP (and optionally further dNTPS like dTTP, dGTP and dCTP) as well as to a kit comprising (in separate compartments) a DpoZ polymerase as described herein-above and dZTP (and optionally further dNTPS like dTTP, dGTP and dCTP). Such a kit may optionally also comprise dATP.

Thus, in summary, the present invention, in particular, relates to the following items.
1. A recombinant nucleic acid molecule comprising a nucleotide sequence encoding a polymerase wherein the polymerase is characterized by the following features:
   (a) it has an amino acid sequence which is at least 50% identical to the amino acid sequence shown in any one of SEQ ID NOs: 1 to 233;
   (b) it does not show 5′-exonuclease activity;
   (c) it accepts 2-amino-2′-deoxyadenosine 5′-triphosphate (dZTP) as a substrate; and
   (d) it does not accept deoxyadenosine 5′-triphosphate (dATP) as a substrate.
2. The recombinant nucleic acid molecule of item 1, wherein the polymerase is furthermore characterized by the feature that it shows 3′-exonuclease activity.
3. The recombinant nucleic acid molecule of item 1, wherein the polymerase is furthermore characterized by the feature that it does not show 3′-exonuclease activity.
4. The recombinant nucleic acid molecule of any one of items 1 to 3, wherein the nucleotide sequence encoding the polymerase is operatively linked to a heterologous promoter sequence.
5. A vector comprising the recombinant nucleic acid molecule of any one of items 1 to 4.
6. A host cell comprising the recombinant nucleic acid molecule of any one of items 1 to 4 or the vector of claim 5.
7. A method for the production of a DNA molecule comprising 2-amino-2′-deoxyadenosine (dZ) instead of deoxyadenosine (dA) comprising the steps of:
   (i) Providing a template DNA molecule to be replicated;
   (ii) Providing dNTPs and wherein said dNTPs include 2-amino-2′-deoxyadenosine-triphosphate (dZTP);
   (iii) Providing a polymerase as defined in any one of claims 1 to 3; and
   (iv) Incubating the components of (i) to (iii).
8. The method of item 7, which furthermore comprises the step of recovering the produced DNA molecule comprising 2-amino-2′-deoxyadenosine (dZ).
9. The method of item 7 or 8 which is carried out in vitro.
10. The method of item 7 or 8 which is carried out in vivo.
11. Use of a polymerase as defined in any one of items 1 to 3 for the production of a DNA molecule comprising 2-amino-2′-deoxyadenosine (dZ) instead of deoxyadenosine (dA).
12. A composition comprising a recombinant nucleic acid molecule of any one of claims 1 to 4, a polymerase as defined in any one of items 1 to 3, a vector of item 5 or a host cell of item 6 and dZTP (and optionally further dNTPS like dTTP, dGTP and dCTP).
13. A kit comprising a recombinant nucleic acid molecule of any one of items 1 to 4, a polymerase as defined in any one of items 1 to 3, a vector of item 5 or a host cell of item 6 and dZTP (and optionally further dNTPS like dTTP, dGTP and dCTP)
14. A polymerase encoded by the nucleic acid molecule of item 3.
15. The polymerase of item 14 characterized in that it contains in its amino acid sequence a substitution at the position which corresponds to position D424 of the amino acid sequence of E. coli pol I (SEQ ID NO: 234), preferably a substitution by alanine.

### FIGURE LEGENDS

FIG. 1: Multiple sequence alignment of DNA polymerases from E. coli (PolA; SEQ ID NO: 234) and Siphoviridae bacteriophages (DpoZ) obtained with the software MUSCLE (default parameter values).
E. coli Pol I (SEQ ID NO: 234)
Acinetobacter phage SH-Ab15497 (SEQ ID NO: 113)
Salmonella phage PMBT28 (SEQ ID NO: 114)
Vibriophage phiVC8 (SEQ ID NO: 112)
Alteromonas phage ‘ZP6 (SEQ ID NO: 117)
Hiyaa phage (SEQ ID NO: 120)
Ghobes phage (SEQ ID NO: 116)
Wayne phage (SEQ ID NO: 115)
Goodman phage (SEQ ID NO: 118)
Theresita phage (SEQ ID NO: 119)

FIG. 2: Detection of 2-amino-2′-deoxyadenosine (dZ) in the genome DNA from bacteriophages.
A: Pattern of restriction endonuclease cleavage obtained with DNA from bacteriophages. Predicted profiles are shown on the left and observed profiles on the right. For Vibrio phage PhiVC8, restriction enzymes with A in site DraI, NcoI, NdeI, NheI are inefficient. For Gordonia phage Ghobes: Enzymes with A in site HindII, BglII, NcoI are inefficient. For Arthrobacter phage Wayne: Enzymes with A in site SalI, HindIII are inefficient but BamHI and XhoI are efficient.
B: HPLC profiles of the nuclease and phosphatase digests obtained with genome DNA from bacteriophages.

FIG. 3: Discrimination between aminoadenine and adenine by siphoviral DpoZ polymerases on homopolymer templates.
The primer extension assays were performed on homopolymer templates using purified versions of His-tagged polymerases produced from the genes encoding the DpoZ phage enzymes or the PolA Klenow fragment of E. coli. Polymerase activity was tested for its ability to elongate a fluorescent (FAM) labeled primer annealed to an unlabeled template.
A: Sequences of primer-template duplexes used for DNA synthesis and chemical nature of the dNTP substrate added to the reaction mix. Z nucleotides contain the 2-aminoadenine base. FAM-labelled primer: X1903 (sequence shown in Table 3). Template: X1904 (sequence shown in Table 3).
B: Polymerization products loaded on denaturing 17% polyacrylamide gels. Lane numbers refer to the primer-template pair and dNTP indicated in A. The gels show extension products synthesized by DNA polymerase for 30 minutes at 37° C.

FIG. 4: Exonuclease activity of DpoZ polymerases on DNA single-strands and double-strands.
A: A 5′-labeled single-strand DNA template (X1459; sequence shown in Table 3) was incubated 5 or 60 minutes at 37 C (in absence of dNTP substrate) with a wild-type DNA polymerase or a 3′-exonuclease-disabled mutant thereof. Reaction products were loaded on denaturing 17% polyacrylamide gels.
B: A 5′-labeled primer (X1903; sequence shown in Table 3) was annealed to an unlabeled template (X1904; sequence shown in Table 3) and the resulting duplex was incubated 5 or 60 minutes at 37 C (in absence of dNTP substrate) with a wild-type DNA polymerase or a 3′-exonuclease-disabled mutant thereof. Reaction products were loaded on denaturing 17% polyacrylamide gels.

FIG. 5: Discrimination between aminoadenine and adenine by siphoviral DpoZ polymerases on heteropolymer templates.
A: The assays were performed as in FIG. 4 except that the template corresponded to a sequence of 50 nucleotides from the genome of the bacteriophage SH-Ab 15497.
B: DNA synthesis catalyzed by wild-type DNA polymerases in response to Z-containing template (X2586) and primer (X2587).
C: DNA synthesis catalyzed by wild-type DNA polymerases in response to A-containing template (X2364) and primer (X2365).

D: DNA synthesis catalyzed by 3′-exonuclease-disabled mutant DNA polymerases in response to Z-containing template (X2586) and primer (X2587). The sequences of the templates and primers are shown in Table 3.

FIG. 6: Alignments produced by the MAFFT software with the sequences of the DpoZ family proteins. The positions indicated by an arrow correspond to the phylogenetically informative sites selected by the BGME software (Default parameters of MAFFT: gap extension penalty: 0.123; gap opening penalty: 1.53; matrix: blosum62. Default parameters of BMGE: matrix: blosum62; sliding window size: 3; maximum entropy threshold: 0.5; gap-rate cutoff: 0.5; minimum block size: 5).
Acinetobacter phage SH-Ab15497 (SEQ ID NO: 113)
Salmonella phage PMBT28 (SEQ ID NO: 114)
Vibriophage ‘phiVC8 (SEQ ID NO: 112)
Alteromonas phage ‘ZP6 (SEQ ID NO: 117)
Hiyaa phage (SEQ ID NO: 120)
Ghobes phage (SEQ ID NO: 116)
Wayne phage (SEQ ID NO: 115)
Goodman phage (SEQ ID NO: 118)
Theresita phage (SEQ ID NO: 119)

FIG. 7: Comparison of active sites between bacterial PolA and siphoviral DpoZ. Perfect conservation of amino acids is observed at the catalytic positions of the 3′-exonuclease domain and of the A motif in the polymerase palm domain, while the B and C motifs are subject to more variations (Loh and. Loeb, DNA Repair. 4, 1390-1398 (2005)).

FIG. 8: Discrimination between dGTP and dITP by siphoviral DpoZ polymerases on a polydC homopolymer template. Polymerase activity was tested for its ability to elongate a fluorescent (FAM) labeled primer (X1903; see Table 3 for the sequence) annealed to an unlabeled template (X1929; sequence shown in Table 3).

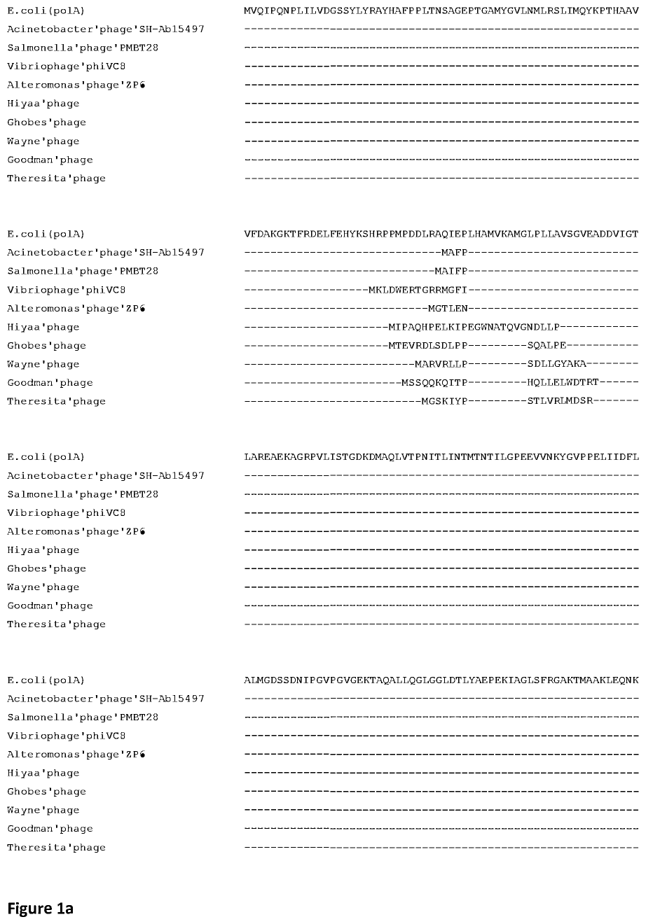

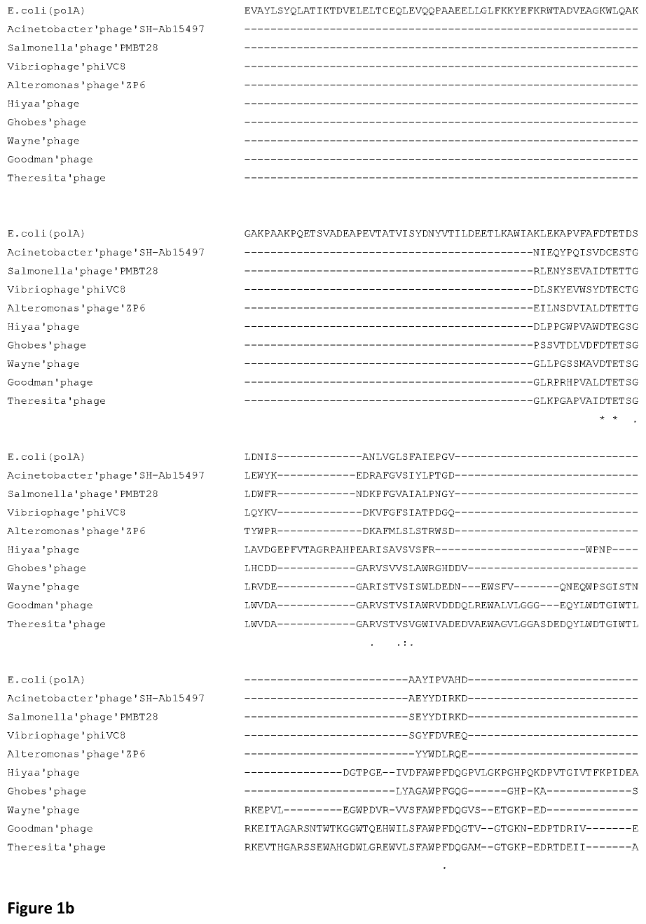

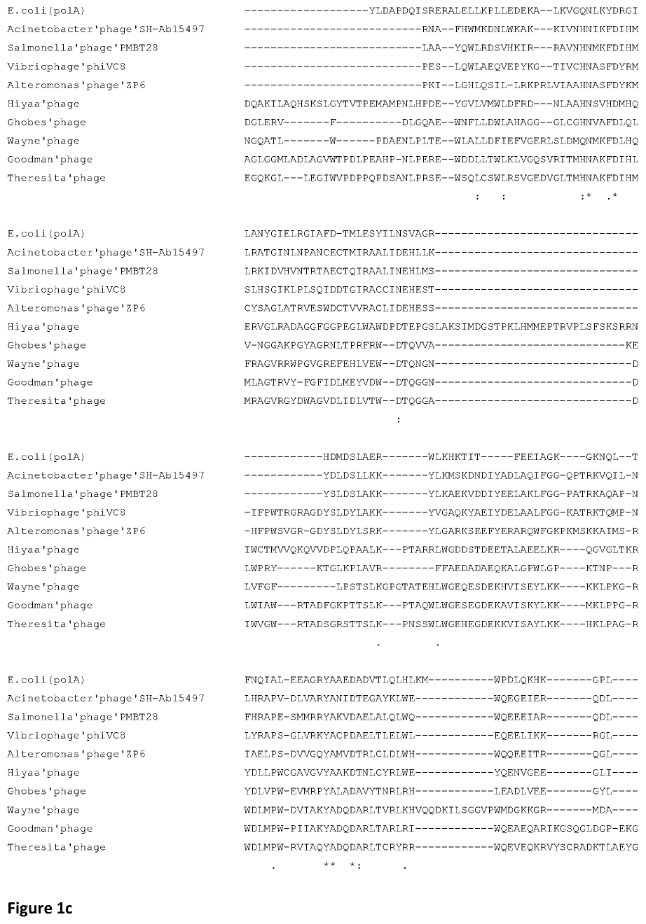

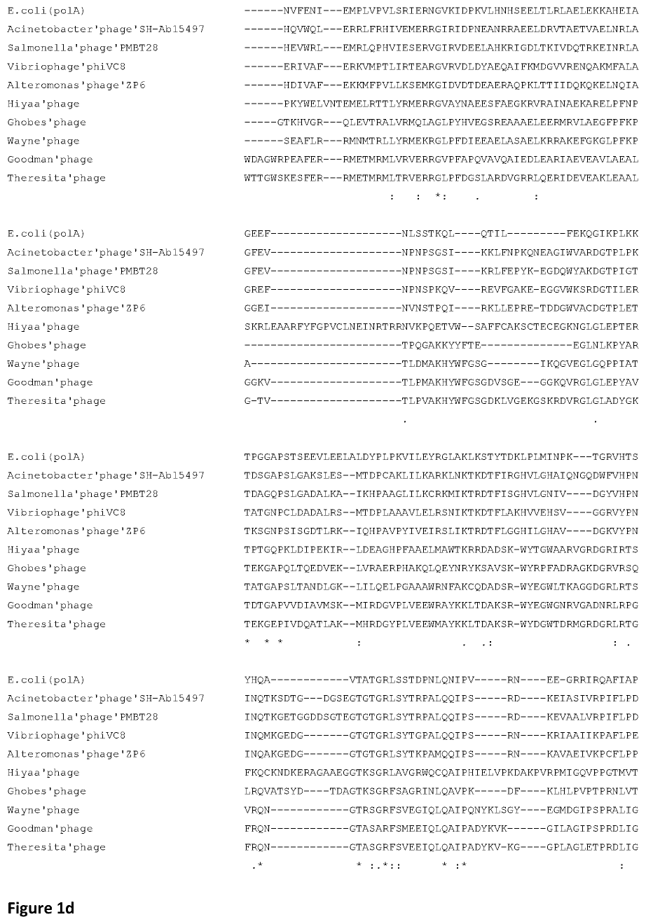

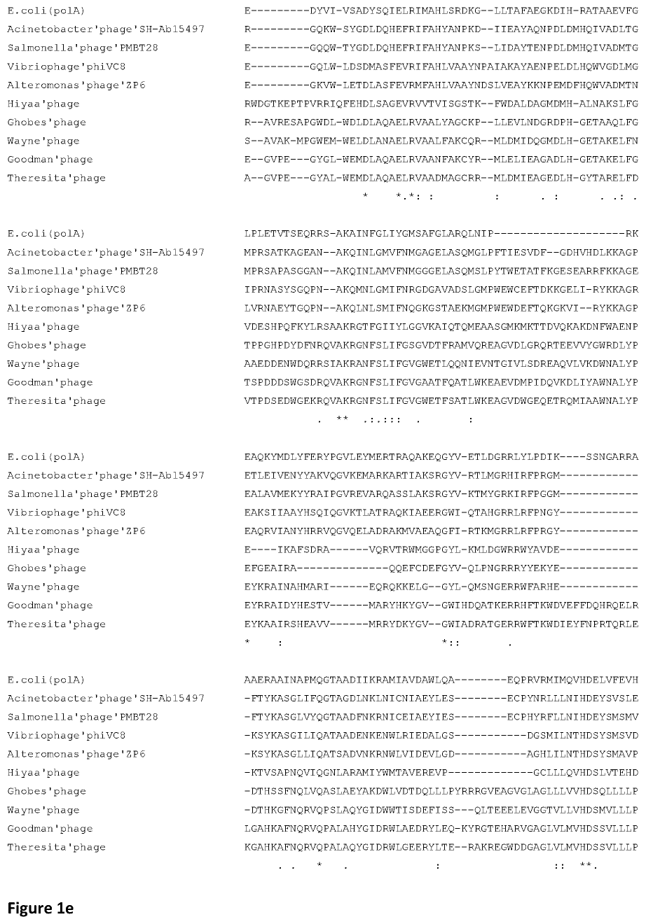

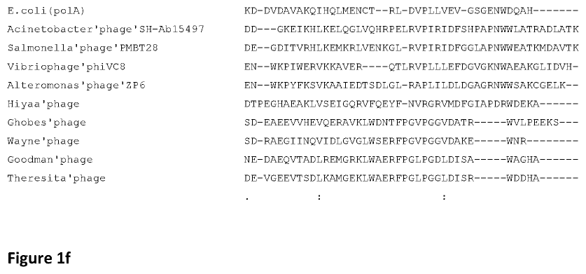

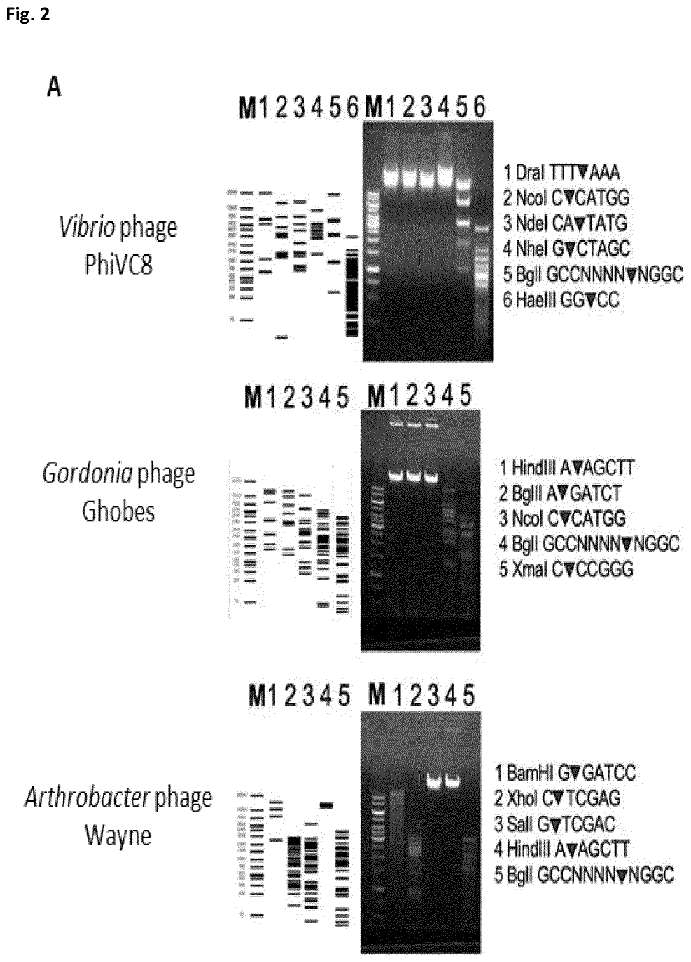

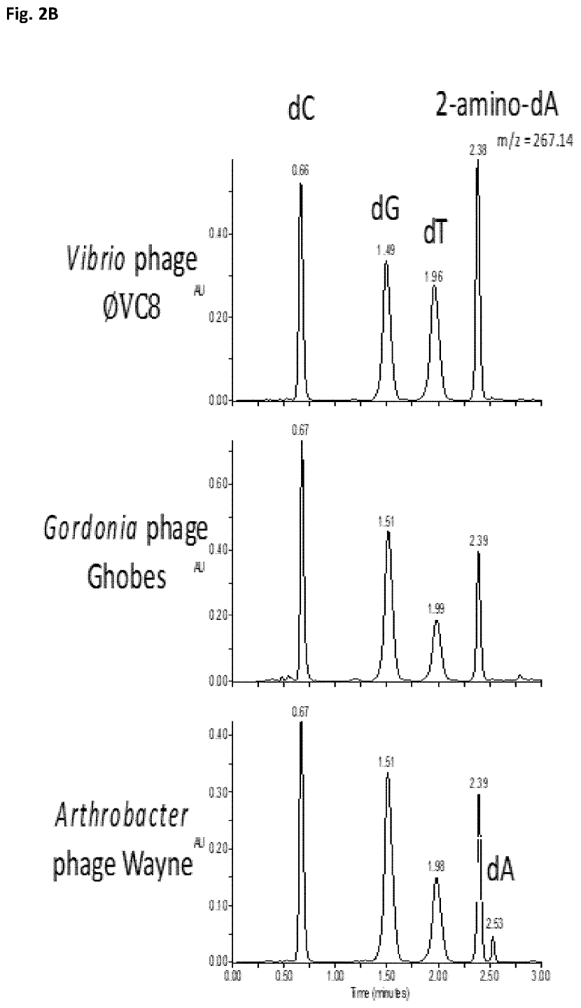

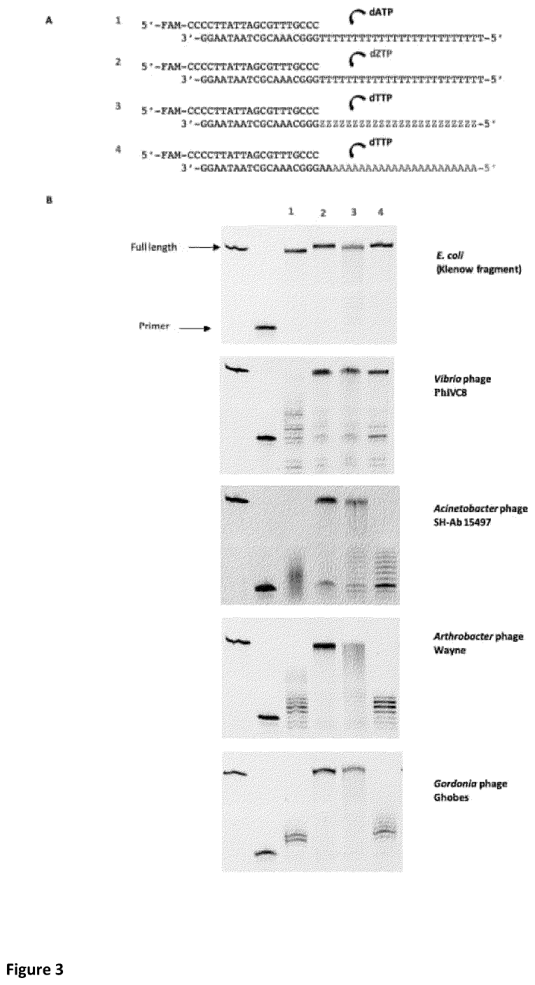

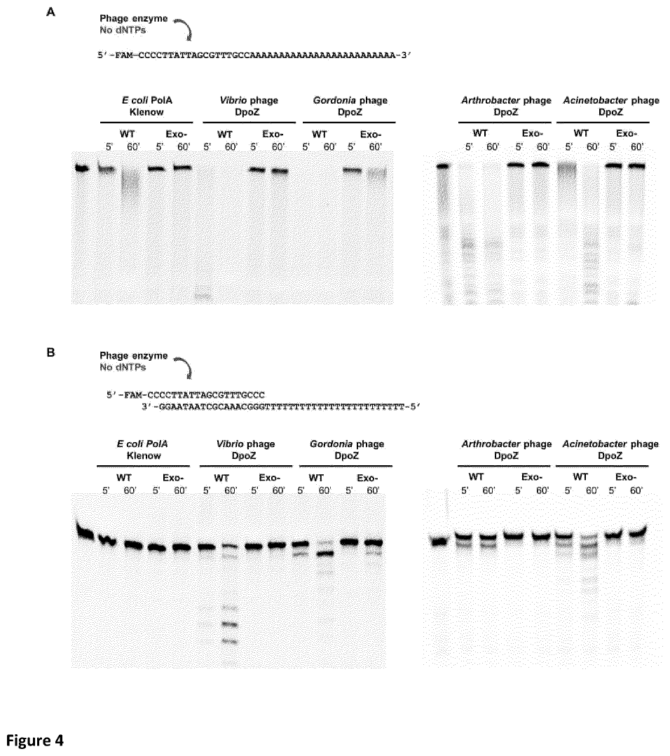

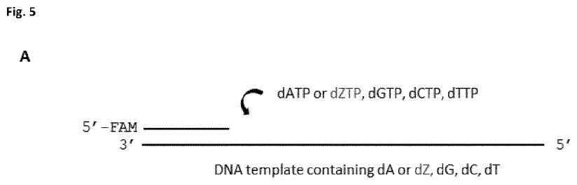

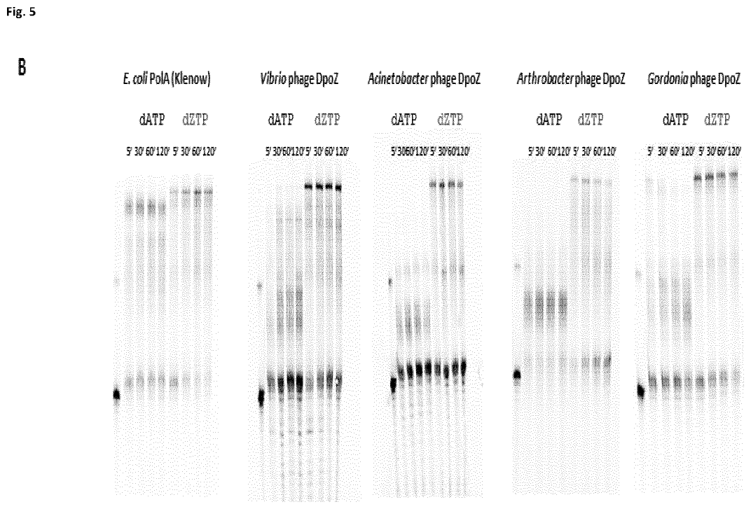

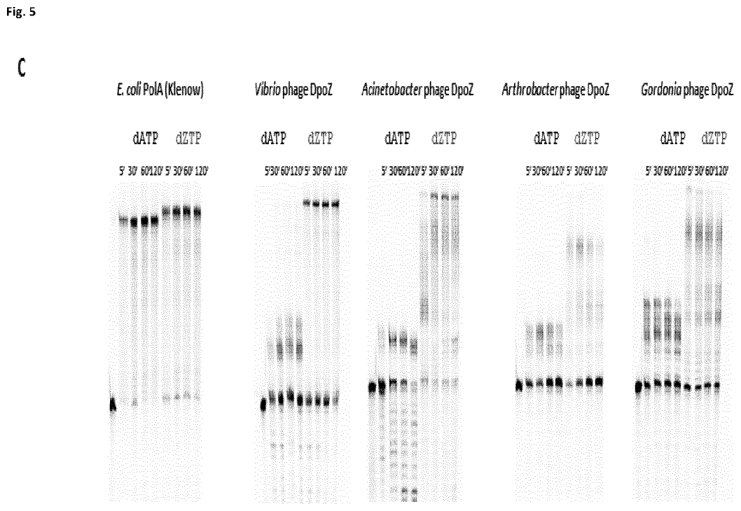

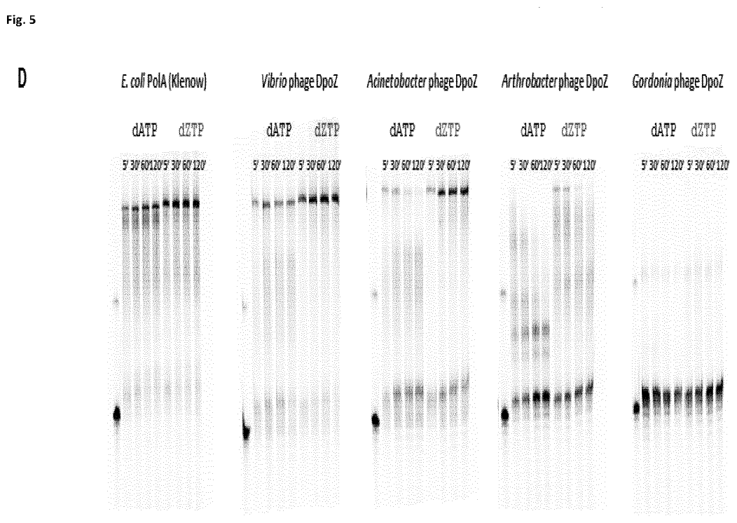

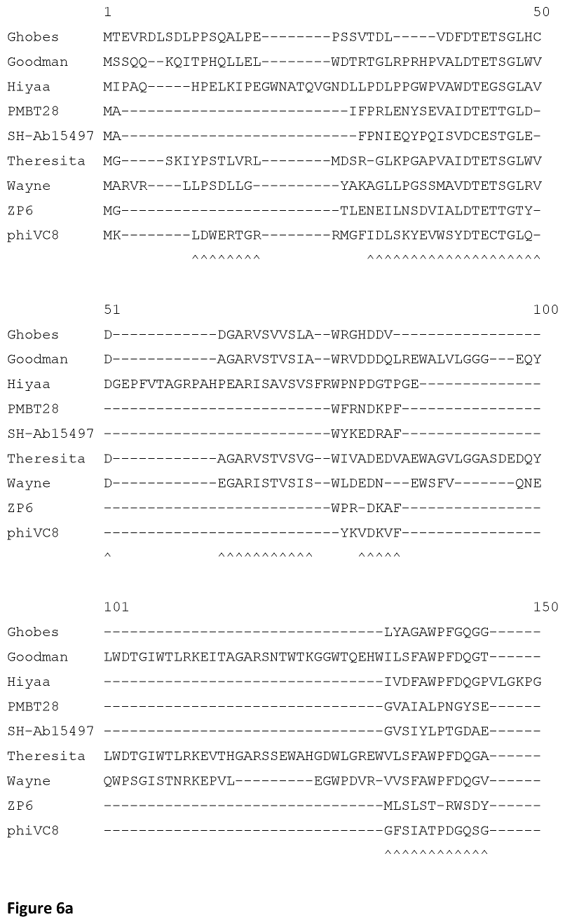

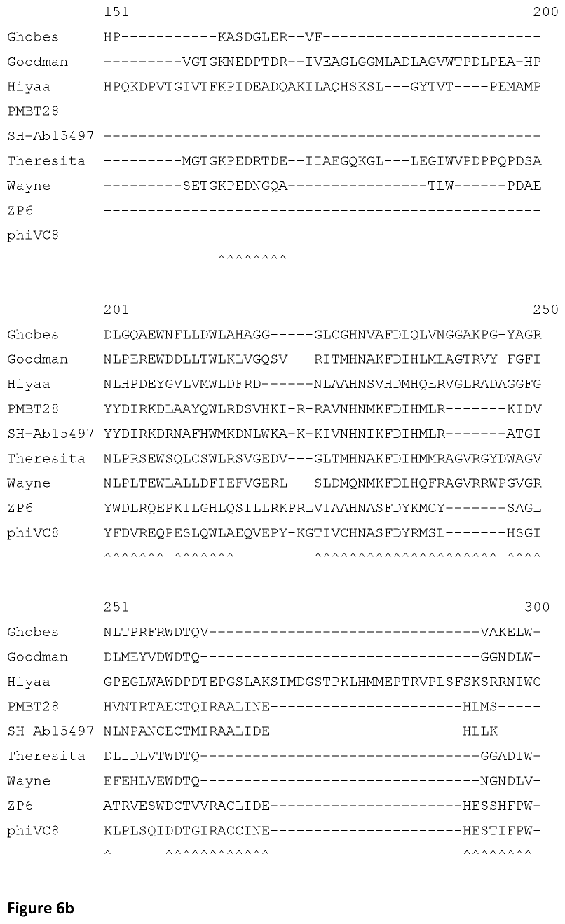

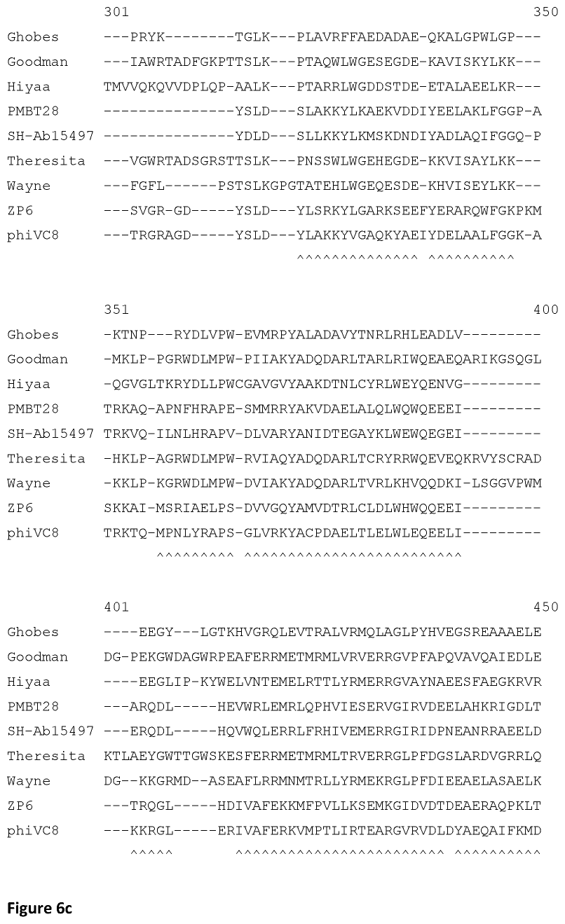

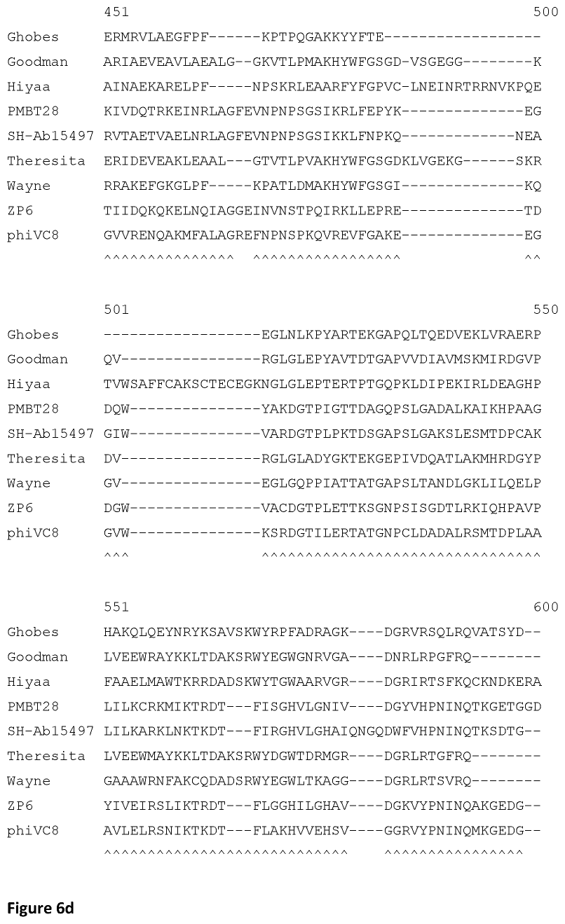

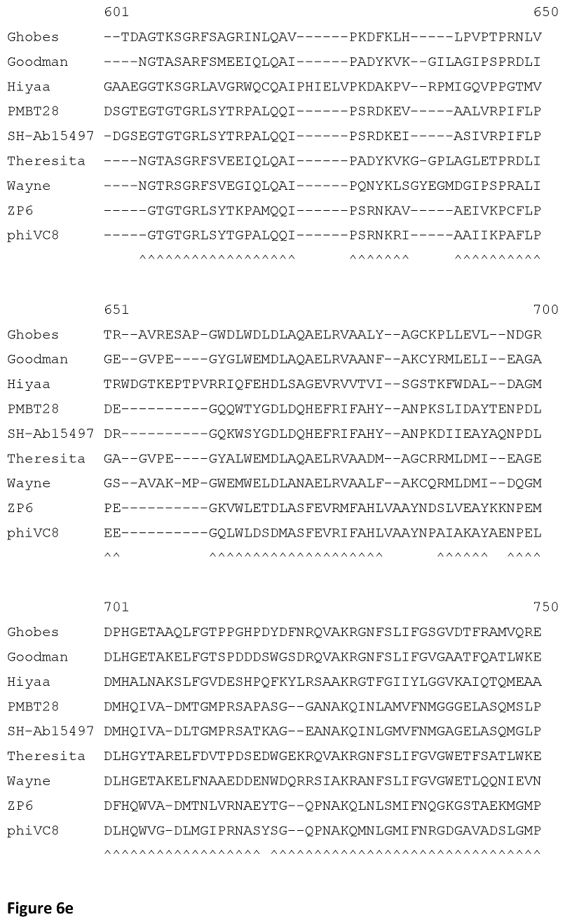

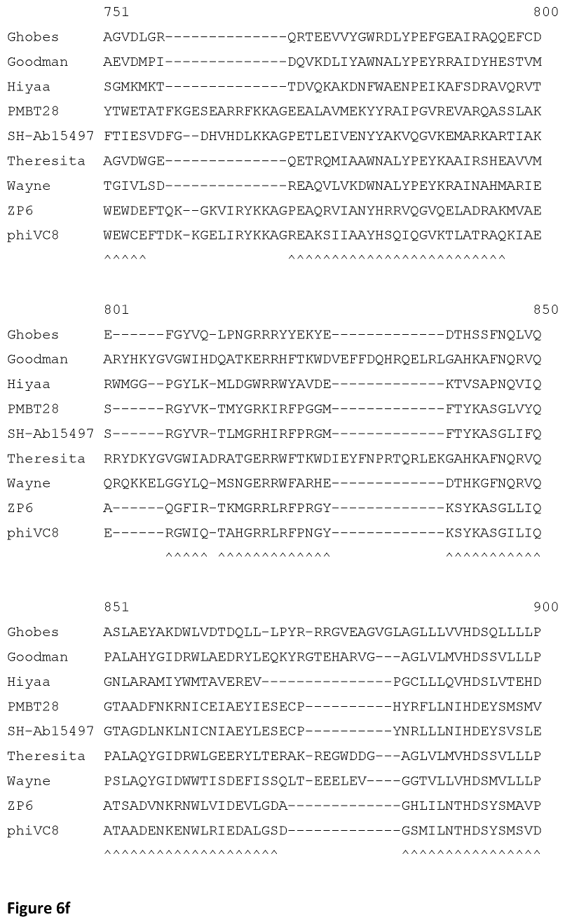

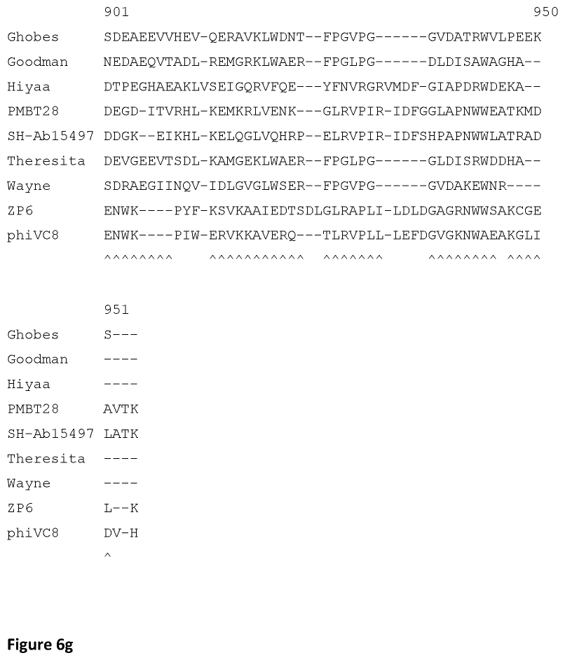

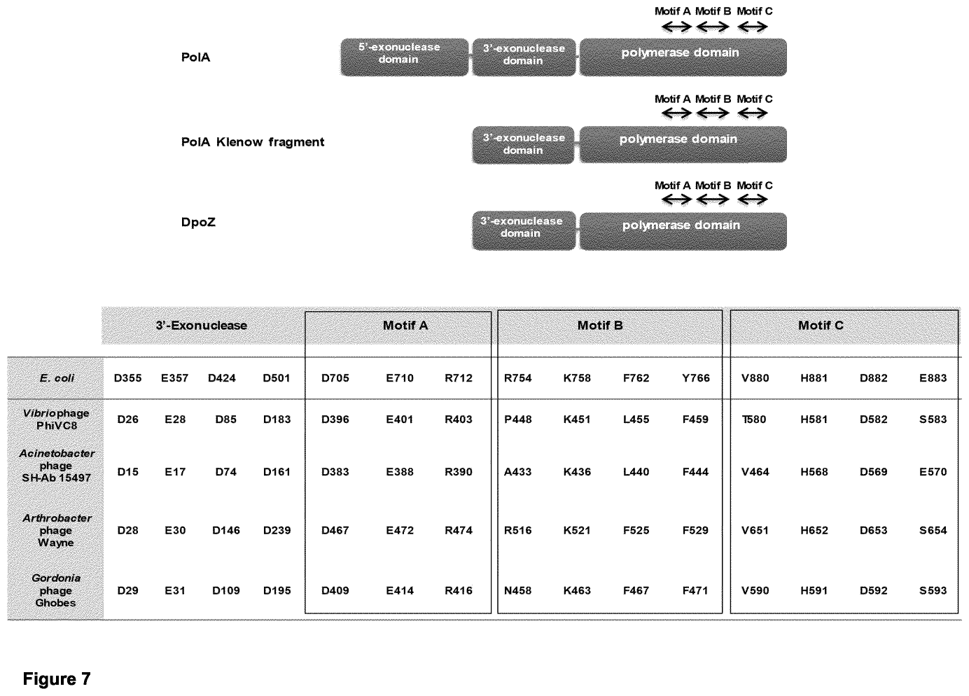

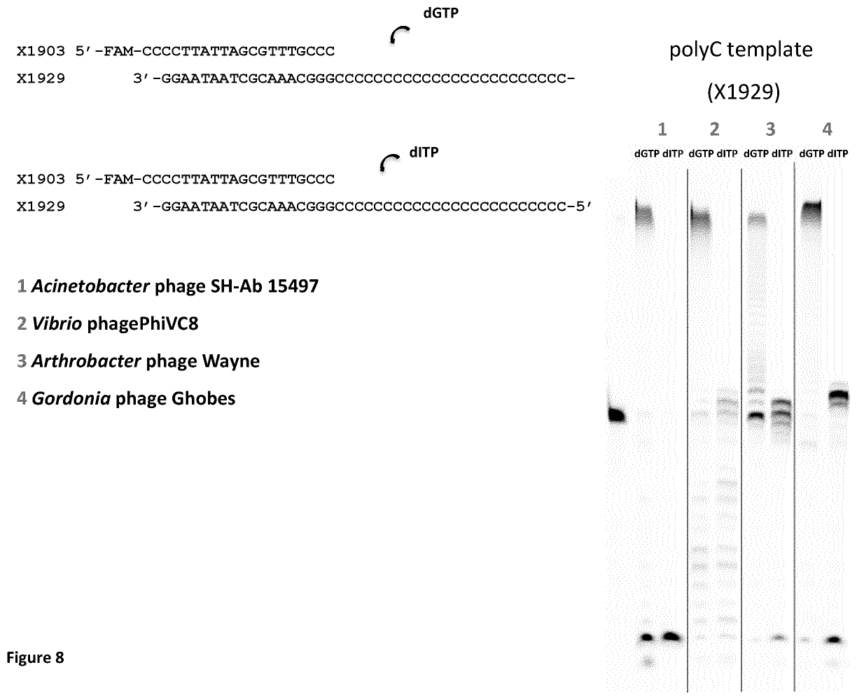

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLES

In the Examples, the following materials and methods were employed.

### Materials and Methods

### Chemicals, Oligonucleotides and Culture Medium

Chemicals were purchased from Sigma Aldrich (Tris HCl, MgCl₂, NaCl, glycerol), NEB biolabs (BSA), Biosolve (DTT) and dNTPs from Invitrogen. Oligonucleotides were synthesized by Eurofins Genomics, their sequences are listed in Table 3. Bacteria were routinely grown in Luria-Bertani medium (LB) at 37° C. When required, antibiotics were added at the following concentrations: 25 mg/L chloramphenicol and 30 mg/L kanamycin.

### Phylogenetic Study

Evolution of the purZ and dpoZ protein families was investigated by reconstructing the phylogenetic trees relating their sequences, translated from the genomes of the ten bacteriophages included in the study. From each set of orthologs, a multiple alignment was computed using the software MAFFT (Katoh and Standley, Molecular Biology and Evolution. 30, 772-780 (2013)) with default parameters. Phylogenetically informative regions were selected in each multiple sequence alignment using the software BMGE with default parameters and retained for downstream analysis. Using the software MrBayes (Huelsenbeck and Ronquist, Bioinformatics. 17, 754-755 (2001)), under a GTR model (Benner et al., Bioinformatics 30 (2014); DOI: 10.1093/bioinformatics/btu461), one million samples were generated from the posterior distribution of the trees relating PolZ (respectively DpoZ) using a parallel Monte Carlo Markov Chain algorithm with 3 parallel chains initialized with an unresolved star tree. After discarding the first 100,000 samples for each of the two phylogenetic trees and retaining every 1000th sample to eliminate auto-correlation, the respective posterior averages of the two sample sets were computed using the software TFBayes (Benner et al., Bioinformatics. 30, i534-i540 (2014)). Final processing was performed with the software FigTree, using mid-point rooting and rotating edges to produce an identical ordering of the species included in both trees.

### Purification of Phage DNA

PhiVC8: 8 billion V. cholerae 01 (Mexico) cells were infected with 80 billion phages in 1 L of LB supplemented with 10 mM CaCl₂) for 16h at 37° C. The cellular debris were centrifuged at 10′000 g for 20 min at 4° C. The supernatant was filtered through a 0.22 μM membrane and the phage particles were precipitated by adding PEG 8000 10% and NaCl 1M at 4° C. for 16h. The mixture was centrifuged at 16′000 g for 20 min at 4° C. and the pellet was resuspended in 50 mM Tris pH 7.4, 100 mM NaCl, 50 mM MgSO₄ before loading a cesium chloride step gradient (from 1.3 to 1.6 g/mL). Pure phages were recovered after a centrifugation at 100′000 g for 16h at 4° C. in a SW41 Rotor. The phages were dialyzed two times against 100 mM Tris pH7.4, 3M NaCl for 16h at 4° C. and once against 100 mM Tris pH7.4, 100 mM NaCl, 50 mM MgSO₄. DNA was prepared by phenol chloroform extraction and ethanol precipitation. Wayne and Ghobes: Phages were amplified by 30-Plate Infection, harvested and concentrated as described above. After centrifugation for 10 minutes at 5′500 g at 4° C., phage pellets were resuspended in about 4-6 mL CaCl₂ buffer solution. CsCl was added to obtain a phage density of 1.5 g/mL and centrifuged at 38′000 rpm for 16 hours. Phages were collected and dialyzed against phage buffer at 4° C. DNA was prepared from phage suspensions by phenol chloroform extraction and ethanol precipitation. Protocols are detailed in the section of the actinobacteriophage database: https://phagesdb.org/workflow/Enzymatic hydrolysis of DNA and analysis of the digests by LC-MS were performed essentially as described by Crain (Methods in Enzymology (Elsevier, 1990)).

### Cloning and Purification of DNA Polymerases

DNA polymerases from Vibrio phage PhiVC8 (AEM62926.1) and from Arthrobacter phage Wayne (ARE89872.1) were amplified from their genomic DNA using as primer couples of oligonucleotides X1168/X1169 and X1840/X1841, respectively. Amplicons were digested with PacI and NotI endonucleases and ligated with a plasmid pGEN452 digested by PvuI and NotI. The vector pGEN452 is a derivative of pet47b plasmid (Novagen) whose MCS has been changed between SaclI and AvrlI to the following sequence 5′-CCGCGGCCCGATCGCCGCGCGGCCGCAAGCTTCCTAGG-3′ (SEQ ID NO: 235).

Synthetic genes encoding DNA polymerases from Acinetobacter phage SH-Ab-15497 (AUG85479.1) and Gordonia phage Ghobes (YP_009281142.1) were obtained from Eurofins Genomics and cloned in the pGEN452 vector. All these plasmids featured an N-terminus of 6 His residues and were used to transform the BL21 C43 strain (Sigma) for protein production. Cultures were grown in LB medium up to an OD (600 nm) of about 0.3 then induced by adding 0.5 mM IPTG and incubated for 16 h at 16° C. Cells were pelleted and frozen overnight at −20° C. Pellets were then lyzed in buffer containing 50 mM NaH2PO4 pH 8, 300 mM NaCl, 1 mM DTT, using lyzonase (Sigma) for 20 min at 30° C. and finally subjected to sonication. The lysate was centrifuged at 10′000 g for 30 min and the supernatant was applied to Protino Ni-TED columns (Macherey Nagel). The eluted proteins were concentrated on Amicon Centricon (50 kDA) (Millipore).

### Mutagenesis of DNA Polymerases

DpoZ genes from each bacteriophage were also cloned in the P15A plasmid pVDM18 previously described (Pezo et al., Sci Rep. 3, 1359 (2013)). Exonuclease mutants were constructed by site directed mutagenesis of pVDM18 Pol constructs using X1379/X1380 oligonucleotides for Vibrio phage PhiVC8 DNA pol (D85A mutation), X1865/X1866 for Arthrobacter phage Wayne DNA pol (D146A mutation), X1990/X1991 for Acinetobacter phage SH-Ab-15497 (D74A mutation), X1867/X1868 for Gordonia phage Ghobes DNA pol (D109A mutation) and X1601/X1602 for Klenow fragment of E. coli DNA pol I (D101A mutation). Mutations were verified by full sequencing of the constructs. These mutants were also sub-cloned into the pGEN452 vector downstream a His6-tag for protein expression

### Primer Extension Assays Templated by Homopolymers or Heteropolymers

Primer extension assays were carried out following a published protocol (Wynne et al., PLoS ONE. 8, e70892 (2013)). Each reaction mix consisted of 100 μL volume containing 1 μM of the FAM-fluorescent-labeled 20-mer primer X1903, 3 μM of the 42-mer template (FIG. 3), 20 mM Tris-HCl (pH 7,5) buffer, 200 mM NaCl (except for Wayne DNA polymerase 50 mM NaCl), 1 mM DTT, 5 mM MgCl₂ and 1 mM dNTP. Primer:template duplexes were annealed by denaturation 2 min at 95° C., and cooled down to 4° C. (0.1° C./s). Polymerisation assay was started by adding 0,034 μM of DNA polymerases after 5 min of primer-template duplexes pre-heating at 37° C. Reactions were incubated at 37° C. for 30 min and 10 μL of reaction were mixed to 10 μL of quenching buffer (98% formamide, 10 mM EDTA). Extension products were separated by electrophoresis migration in denaturing polyacrylamide gels (7M, 1×TBE, 17% acrylamide gel). Gels were analyzed using the G-BOX (Ozyme) and band intensities were quantified with Genetools software (Syngene).

### Determination of Kinetic Parameters of DNA Polymerases

Kinetic parameters were determined in a single nucleotide extension assay using DNA polymerase mutants devoid of 3′exonuclease activity, followed by denaturing polyacrylamide gel electrophoresis of the extended products essentially as described (O'Flaherty and Guengerich, Current Protocols in Nucleic Acid Chemistry. 59 (2014)). The fluorescent oligonucleotide X1648 was annealed to X1649 template. Duplex substrates were used at 500 nM concentration in reaction buffer (20 mM TrisHCl pH7, 200 mM NaCl, 2 mM DTT, 5 mM MgCl2), with different enzymes concentrations and various dNTPs concentrations. Reactions were performed at 37° C. for variable times in conditions to remain in the confines of the steady-state kinetic model e.g. remaining below 20% of product formation (Creighton et al., Methods in Enzymology (Elsevier, 1995; https://linkinghub.elsevier.com/retrieve/pii/0076687995620214), vol. 262, pp. 232-256).

### Example 1: Identification of a New Functional Class of Polymerases, the DpoZ Family

Protein databases (Uniprot) were searched using the PurZ sequence from phage S-2L as query. In this search a cluster of homologs of the PurZ sequence was identified with all identified sequences belonging to the Siphoviridae bacteriophages (Siphoviruses). The viruses of this cluster infect cellular hosts as distant as Gram-negative proteobacteria (Vibrio, Salmonella and Acinetobacter) Gram-positive actinobacteria (Arthrobacter and Gordonia) and cyanobacteria (Synechococcus) and dwell habitats as diverse as soil, freshwater and seawater. A summary of the results is shown in Table 1.

TABLE 1

| | | | | | PurZ | DpoZ |
|---|---|---|---|---|---|---|
| Case | | | Size | | (PurA | (PolA |
| studied | Species | Genome | kbp | G + C % | paralog) | paralog) |
| X | Synechococcus phage S-2L | AX955019.1 | 45.57 | 69.4 | 1 | |
| X | Vibrio phage phiVC8 | JF712866.1 | 39.42 | 50.8 | 1 | 1 |
| | Vibrio phage QH | KM612259.1 | 39.73 | 50.5 | 1 | 1 |
| | Vibrio phage JSF15 | KY883642.1 | 39.64 | 50.7 | 1 | 1 |
| | Vibrio phage CJY | KM612260.1 | 39.54 | 50.6 | 1 | 1 |
| | Vibrio phage H2 SGB-2014 | KM612262.1 | 39.53 | 50.5 | 1 | 1 |
| | Vibrio phage J2 | KM612264.1 | 39.53 | 50.6 | 1 | 1 |
| | Vibrio phage H1 | KM612261.1 | 39.53 | 50.5 | 1 | 1 |
| | Vibrio phage H3 | KM612263.1 | 39.53 | 50.5 | 1 | 1 |
| | Vibrio phage J3 | KM612265.1 | 39.78 | 50.5 | 1 | 1 |
| | Vibrio phage Rostov 6 | MH105773.1 | 39.93 | 50.7 | 1 | 1 |
| | Vibrio phage JSF33 | KY883647.1 | 39.66 | 50.6 | 1 | 1 |
| | Vibrio phage VP5 | AY510084 | 39.78 | 50.5 | 1 | 1 |
| | Vibrio phage VP2 | AY5505112 | 39.85 | 50.6 | 1 | 1 |
| | Podoviridae sp. ctpVR23 | MN582112.1 | 37.41 | 49.4 | 1 | 1 |
| | Alteromonas phage ZP6 | MK203850.1 | 37.74 | 50.1 | 1 | 1 |
| | Ochrobactrum phage vBOspPOH | MT028492.1 | 41.23 | 55.2 | 1 | 1 |
| X | Acinetobacter phage SH-Ab 15497 | MG674163.1 | 43.42 | 47.9 | 1 | 1 |
| | Salmonella phage PMBT28 | MG641885.1 | 48.11 | 58.6 | 1 | 1 |
| | Siphoviridae sp. | MH622939.1 | 48.74 | 61.7 | 1 | 1 |
| | Bacillus phage vB_BpsS-140 | MH884512.1 | 55.09 | 39.8 | 1 | |
| X | Arthrobacter phage Wayne | KU160672.2 | 44.37 | 61.1 | 1 | 1 |
| | Arthrobacter phage Litotes | MK279863.1 | 43.5 | 61.1 | 1 | 1 |
| | Arthrobacter phage AppleCider | MN735429.1 | 43.7 | 61.1 | 1 | 1 |
| | Arthrobacter phage CallieOMalley | MK112535.1 | 43.86 | 61.2 | 1 | 1 |
| | Arthrobacter phage Suppi | KX621004.1 | 43.91 | 61.2 | 1 | 1 |
| | Arthrobacter phage Canowicakte | MF140400.1 | 43.91 | 61.2 | 1 | 1 |
| | Arthrobacter phage DreamTeam | MK919484.1 | 43.09 | 60.7 | 1 | 1 |
| | Arthrobacter phage Bennie | KU160640.2 | 43.08 | 61.4 | 1 | 1 |
| | Arthrobacter phage MeganNoll | MG198782.1 | 44.26 | 60.9 | 1 | 1 |
| | Arthrobacter phage Korra | KU160653.2 | 43.71 | 61.1 | 1 | 1 |
| | Arthrobacter phage BigMack | MK112529.1 | 43.36 | 61.3 | 1 | 1 |
| | Arthrobacter phage Vallejo | KX621005.1 | 43.61 | 61.0 | 1 | 1 |
| | Arthrobacter phage Dino | MF140407.1 | 43.56 | 61.1 | 1 | 1 |
| | Arthrobacter phage Fluke | MG198781.1 | 43.81 | 60.9 | 1 | 1 |
| | Arthrobacter phage Carpal | MK112538.1 | 43.71 | 61.0 | 1 | 1 |
| | Arthrobacter phage Beethoven | MK112528.1 | 43.93 | 61.0 | 1 | 1 |
| | Arthrobacter phage Immaculata | KU160649.1 | 43.66 | 61.0 | 1 | 1 |
| | Arthrobacter phage Cholula | MK279845.1 | 43.66 | 61.0 | 1 | 1 |
| | Arthrobacter phage Rozby | MK279876.1 | 43.2 | 61.0 | 1 | 1 |
| | Arthrobacter phage Wawa | MK279892.1 | 43.85 | 60.9 | 1 | 1 |
| | Arthrobacter phage Potatoes | MK279901.1 | 43.66 | 61.0 | 1 | 1 |
| | Arthrobacter phage MrGloopy | MK660714.1 | 43.7 | 60.9 | 1 | 1 |
| | Arthrobacter phage Scuttle | MK814749.1 | 43.73 | 61.1 | 1 | 1 |
| | Arthrobacter phage Riverdale | MK279875.1 | 43.39 | 61.0 | 1 | 1 |
| | Arthrobacter phage TattModd | MK279886.1 | 43.55 | 61.0 | 1 | 1 |
| | Arthrobacter phage HeadNerd | MK279907.1 | 42.97 | 61.4 | 1 | 1 |
| | Arthrobacter phage RAP15 | KU160662.1 | 44.26 | 60.9 | 1 | 1 |
| | Arthrobacter phage Savage2526 | MK279880.1 | 43.42 | 61.0 | 1 | 1 |
| | Arthrobacter phage DrRobert | KU160643.1 | 42.6 | 60.6 | 1 | 1 |
| | Arthrobacter phage PitaDog | MF140425.1 | 42.96 | 60.7 | 1 | 1 |
| | Arthrobacter phage Glenn | KU160645.1 | 44.39 | 60.8 | 1 | 1 |
| | Arthrobacter phage Zorro | MK279896.1 | 43.56 | 61.1 | 1 | 1 |
| | Arthrobacter phage Lucy | KX576641.1 | 42.94 | 60.7 | 1 | 1 |
| | Arthrobacter phage Moki | MH744421.1 | 43.16 | 61.3 | 1 | 1 |
| | Arthrobacter phage Huckleberry | MK279856.1 | 42.97 | 61.4 | 1 | 1 |
| | Arthrobacter phage ChewChew | MK279844.1 | 43.44 | 60.7 | 1 | 1 |
| | Arthrobacter phage Pterodactyl | MK279872.1 | 43.27 | 60.8 | 1 | 1 |
| | Arthrobacter phage Christian | MF140404.1 | 43.08 | 60.7 | 1 | 1 |
| | Arthrobacter phage Preamble | KU160659.1 | 43.37 | 60.7 | 1 | 1 |
| | Arthrobacter phage Lennox | MK279862.1 | 43.09 | 60.7 | 1 | 1 |
| | Arthrobacter phage Bodacious | MK112531.1 | 43.24 | 60.7 | 1 | 1 |
| | Arthrobacter phage CristinaYang | MK279847.1 | 43.37 | 60.7 | 1 | 1 |
| | Arthrobacter phage Lasagna | MK279860.1 | 42.6 | 60.8 | 1 | 1 |
| | Arthrobacter phage Nancia | MK279867.1 | 43.24 | 60.7 | 1 | 1 |
| | Arthrobacter phage LilStuart | MN813680.1 | 42.95 | 60.7 | 1 | 1 |
| | Arthrobacter phage OurGirlNessie | MK279869.1 | 42.56 | 60.7 | 1 | 1 |
| | Arthrobacter phage Aledel | MK112526.1 | 43.74 | 61.6 | 1 | 1 |
| | Arthrobacter phage Eunoia | MK279851.1 | 44.42 | 61.5 | 1 | 1 |
| | Arthrobacter phage OMalley | MK279868.1 | 43.74 | 61.6 | 1 | 1 |
| | Arthrobacter phage Riovina | MK279874.1 | 43.74 | 61.6 | 1 | 1 |
| | Arthrobacter phage HunterDalle | KU160648.1 | 43.34 | 61.6 | 1 | 1 |
| | Arthrobacter phage Vulture | KU160671.1 | 43.34 | 61.6 | 1 | 1 |
| | Arthrobacter phage Supakev | MK279884.1 | 43.76 | 61.7 | 1 | 1 |
| | Arthrobacter phage EstebanJulior | MK919476.1 | 43.75 | 61.3 | 1 | 1 |
| | Arthrobacter phage Urla | MG198779.1 | 43.94 | 61.2 | 1 | 1 |
| | Arthrobacter phage Pumancara | KU160661.1 | 42.83 | 61.7 | 1 | 1 |
| | Arthrobacter phage Joann | KU160652.1 | 44.18 | 60.7 | 1 | 1 |
| | Arthrobacter phage Greenhouse | KX688103.1 | 43.98 | 60.8 | 1 | 1 |
| | Arthrobacter phage Temper16 | MF668285.1 | 43.95 | 61.9 | 1 | 1 |
| | Arthrobacter phage Daiboju | MH450117.1 | 43.95 | 61.9 | 1 | 1 |
| | Arthrobacter phage Herb | MH450118.1 | 43.95 | 61.9 | 1 | 1 |
| | Arthrobacter phage KingBob | MH450121.1 | 43.95 | 61.9 | 1 | 1 |
| | Arthrobacter phage Maria1952 | MN586061.1 | 43.95 | 61.9 | 1 | 1 |
| | Arthrobacter phage Sergei | MH450131.1 | 43.95 | 61.9 | 1 | 1 |
| | Arthrobacter phage Oxynfrius | KX688102.1 | 44.16 | 60.8 | 1 | 1 |
| | Arthrobacter phage Nubia | MF140424.1 | 44.05 | 60.7 | 1 | 1 |
| | Arthrobacter phage GreenHearts | MK279854.1 | 44.25 | 60.8 | 1 | 1 |
| | Arthrobacter phage RcigaStruga | KX576640.1 | 43.89 | 60.7 | 1 | 1 |
| | Arthrobacter phage Huntingdon | MG210949.1 | 43.89 | 60.7 | 1 | 1 |
| X | Gordonia phage Ghobes | KX557278.1 | 45.29 | 65.2 | 1 | 1 |
| | Streptomyces phage Hiyaa | MK279841.1 | 83.22 | 66.7 | 1 | 1 |
| | Streptomyces phage Galactica | MT316461.1 | 81.22 | 66.9 | 1 | 1 |
| | Microbacterium phage Sucha | MK737942.1 | 41.83 | 66.1 | 1 | 1 |
| | Microbacterium phage Theresita | MK660713.1 | 40.23 | 65.9 | 1 | 1 |
| | Microbacterium phage Zanella | MN369765.1 | 42.11 | 67.0 | 1 | 1 |
| | Microbacterium phage Goodman | MK016495.1 | 42.36 | 66.3 | 1 | 1 |
| | Microbacterium phage Johann | MK016497.1 | 42.36 | 66.3 | 1 | 1 |
| | Oleibacter marinus | NZ_FT0H00000000 | 3859 | 53.1 | 2 | 1 |
| | Desulfobulbus sp | JROS00000000 | 4200 | 59.0 | 2 | 1 |
| | Chondromyces crocatus | CP012159 | 11300 | 68.7 | 3 | |

Strikingly, a gene homologous to polA for DNA polymerase I was found to occur in synteny with purZ in all these phage genomes, but not in S-2L.

Protein sequence alignments revealed that the identified phage polymerase homologs corresponded precisely to the Klenow fragment of E. coli PolA lacking the 5′-exonuclease domain but retaining the 3′-exonuclease domain (FIG. 1). The presumptive polymerase gene from Siphoviruses was designated dpoZ, in accordance with Demerec's nomenclature Genetics 54 (1966), 61-67; see also https://jb.as.org/content/nomenclature). FIG. 6 shows an alignment of 9 of the identified dpoZ genes produced by the MAFFT software.

### Example 2: Comparative Phylogeny of DpoZ and PurZ

The synteny between the dpoZ and purZ genes in the metabolic region of siphoviral genomes suggested that the two functions coexisted in a common ancestor encoded with the Z base and have since coevolved. Thus, a phylogenetic study was conducted to test this hypothesis. A phylogenetic tree was reconstructed separately for each family, by applying the same algorithmic method which consisted in the multiple alignment of sequence families followed by a posteriori average reconstruction using a GTR model (Benner et al., Bioinformatics 30 (2014); DOI: 10.1093/bioinformatics/btu461)). Two almost perfectly congruent unrooted trees were obtained for PurZ and DpoZ.

The slightly different placement of the Hiyaa phage in the two trees is not disturbing given that its genome displays a dislocated synteny and a size twice as large (83 kbp) as that of the other siphoviruses (see Table 1). The Synechococcus phage S-2L represents another special case, as it does not encode a PolA homolog and can therefore only be analyzed using the PurZ tree. It should be noted that the genomic composition of the S-2L phage is the poorest in Z:T pairs of all siphoviruses bearing a purZ gene (see Table 1).

The tree obtained for the PurZ sequences places the S-2L phage in a branch with the phages infecting proteobacteria. This placement makes sense, considering that cyanobacteria and proteobacteria are Gram− while the actinobacterial hosts of other siphoviruses are Gram+. Overall, the phylogenetic study indicated that the base Z has been used as an information carrier among siphoviruses since at least a date prior to the evolutionary divergence between actinobacteria, cyanobacteria and proteobacteria. The paralogous character of sequence homologies within the PurA/PurZ on the one hand, and PolA/DpoZ on the other hand, raises the question of the nature of the information carrier in their common ancestor, either A or Z, at an earlier evolutionary stage.

### Example 3: DNA Composition of DpoZ-Encoding Phages

To ascertain the presence of dZ in siphoviral genomes that include purZ genes and the newly identified dpoZ genes, chemical analysis of phage DNA was applied. Phage DNA was purified as described above in the Material and Methods section. Enzymatic digestion by restriction endonucleases of the genomic DNA from the bacteriophage samples was found to follow cleavage patterns congruent with those reported for S-2L DNA (FIG. 2A). In particular, the restriction enzymes cleaving adenine-containing hexamers did not digest DNA from the Vibrio phage (PhiVC8), Gordonia phage (Ghobes) nor Arthrobacter phage (Wayne), strongly suggesting that they all contain aminoadenine (FIG. 2A).

Chemical evidence for the absence of deoxyadenosine (dA) and presence of 2-aminodeoxyadenosine (dZ) was provided by HPLC fractionation and mass spectrometry applied to enzymatic hydrolysates of phage DNA (FIG. 2B). A minor fraction of dA was found together with dZ in the DNA from the Arthrobacter phage (FIG. 2B).

### Example 4: Analysis Whether dZTP can Act as a Substrate for dpoZ-Encoded Polymerases

The replacement of adenine with aminoadenine in phage DNA, consistent with the synteny of purZ and dpoZ in siphoviral genomes, led to the investigation whether dZTP can act as substrate of dpoZ-encoded polymerases.

### (a) Discrimination Against Adenine and for Aminoadenine

The fact that a DNA polymerase is encoded by Z-containing genomes begs the question whether the viral enzyme DpoZ could discriminate for aminoadenine and against adenine during replication. In this context, experiments of primer extension using simple combinations of Z- or A-containing deoxynucleoside triphosphates and templates were performed as described in the Material and Methods section.

It is shown in FIG. 3 that dZTP leads to the formation of full-length products in response to (dT)24 template using the His-tagged versions of the four phage polymerases produced in E. coli and purified to homogeneity. By contrast dATP leads to incomplete polymerization products under the same conditions.

A similar discrimination effect for Z and against A is also observed when dTTP is reacted by DpoZ enzymes in response to polypurine templates (dA)24 and (dZ)24. Again, a congruent activity is observed for the four siphoviral DpoZ, which form full-length products only when dZTP is provided as substrate (FIG. 3B). Remarkably, the Klenow fragment of PolA from E. coli (His-tagged identically) catalyzes the polymerization of full-length product with all the combinations of substrates and templates, showing little discrimination for or against Z (FIG. 3B).

The coexistence of degradation and polymerization activities in DpoZ and PolA enzymes complicates the precise measurement of kinetic parameters. Thus, it was attempted to disable DNA degradation in DpoZ by mutating the catalytic site of 3′-exonuclease performing the proofreading activity. Corresponding mutants were constructed as described in the Materials and Methods section. As expected, fluorescent primers no longer underwent degradation by the mutant DpoZ enzymes (FIG. 4). However, the mutation inactivating 3′-exonuclease also diminished polymerase activity in the case of the Arthrobacter phage and abolished it in the case of the Gordonia phage. Kinetic parameters were thus measured only using exonuclease-disabled versions of the Vibrio and Acinetobacter phage DpoZ. As shown in Table 2, quantitative confirmation of the discrimination effect qualitatively displayed in FIG. 3 was obtained by measuring the affinity (KM) and turnover number (kcat) of the two DpoZ enzymes compared with Klenow PolA. A higher factor of 90-fold in catalytic efficiency with dZTP relative to dATP was calculated in the case of the Vibrio phage and of 29-fold in the case of the Acinetobacter phage, to be compared with 0.5-fold in the case of the Klenow enzyme.

TABLE 2
Enzymology of the discrimination between dZTP and dATP by siphoviral DNA polymerases.

| DNA | K_{M} (mM) | | K_{M} ^{(A)}/ | k_{cat} (s⁻¹) | | k_{cat}^{(Z)}/ | Efficiency (mM⁻¹ s⁻¹) | | K_{M} ^{(A)} k_{cat}^{(Z)}/ |
|---|---|---|---|---|---|---|---|---|---|
| polymerase | dZTP | dATP | K_{M}^{(Z)} | dZTP | dATP | k_{cat}^{(A)} | dZTP | dATP | K_{M}^{(Z)} k_{cat}^{(A)} |
| E. coli | 0.81 ± 0.14 | 0.70 ± 0.17 | 0.86 | 13.96 ± 1.99 | 20.61 ± 1.52 | 0.68 | 17.73 ± 4.96 | 30.40 ± 5.82 | 0.58 |
| Klenow | | | | | | | | | |
| fragment | | | | | | | | | |
| Vibrio phage | 0.10 ± 0.02 | 4.50 ± 1.38 | 45.00 | 0.42 ± 0.14 | 0.21 ± 0.05 | 2.00 | 4.35 ± 1.56 | 0.048 ± 0.017 | 90.62 |
| PhiVC8 | | | | | | | | | |
| Acinetobacter | 1.30 ± 0.36 | 21.40 ± 3.40 | 16.47 | 0.29 ± 0.07 | 0.16 ± 0.01 | 1.81 | 0.23 ± 0.06 | 0.008 ± 0.001 | 28.75 |
| phage | | | | | | | | | |
| SH-Ab 15497 | | | | | | | | | |

The Km affinity constant and the kcat turnover number of 3′-exonuclease-disabled and His-tagged versions of DpoZ polymerases from Vibrio phage PhiVC8 and Acinetobacter phage SH-Ab 15497 are shown in comparison with PolA polymerase (Klenow fragment) from E. coli. The average and standard deviation of three independent assays carried out under the same conditions are given for each experiment.

To emulate polymerization processes closer to the natural history of the phages, primer extension assays were also conducted using templates corresponding to a 50-mer sequence from the siphoviral genome SH-Ab 1549 (FIG. 5). Judging by the size of elongation products, the dZTP substrate is preferred to dATP by siphoviral DNA polymerases, whether the template contains Z or A (FIGS. 5B and 5C). This result contrasts with Klenow bacterial DNA polymerase, which shows a preference for dATP with A-containing template. Mutant polymerases lacking 3′-exonuclease activity produce longer elongation products with dATP, compared to wild-type DpoZ in the case of the proteobacterial phages PhiVC8 and SH-Ab-15497 (FIG. 5D). As noted previously, only attenuated polymerase activity remains in the DpoZ mutant enzymes lacking 3-exonuclease activity in the case of Ghobes and Wayne actinobacterial siphoviruses (FIG. 5D).

### CONCLUSION

The present invention describes a novel category of DNA polymerases encoded with an alien nucleobase (2-aminoadenosin) and discriminating against the incorporation of the canonical counterpart (adenosine). So far, no cellular polymerase that ostracizes a canonical base had been described. The newly identified polymerases open up new possibilities for chemically diversifying replicons in vivo.

The following oligonucleotides were employed in the above-described Examples:

| | Oligonucleotides |
|---|---|
| X1168 | 5′-GGGTTAATTAATGAAGCTAGATTGGGAAAG |
| | AACAGG-3′ |
| | (SEQ ID NO: 236) |
| X1169 | 5-GGGTCTATGCGGCCGCTTAATGTACATCGA |
| | TTAATCCTTTTGCTTCGG-3′ |
| | (SEQ ID NO: 237) |
| X1840 | 5′-GGGTTATTAATGGCGCGAGTGCGCCTACTG |
| | -3′ |
| | (SEQ ID NO: 238) |
| X1841 | 5′-GGGTCTATGCGGCCGCTTACCGGTTCCATT |
| | CCTTCG-3′ |
| | (SEQ ID NO: 239) |
| X1379 | 5′-GTCATAACGCGTCATTTGCGTACAGAATGT |
| | CG-3′ |
| | (SEQ ID NO: 240) |
| X1380 | 5′-CGACATTCTGTACGCAAATGACGCGTTATG |
| | AC-3′ |
| | (SEQ ID NO: 241) |
| X1865 | 5′-CATGAAGTTCGCGCTGCACCAGTTCCGCGC |
| | TGG-3′ |
| | (SEQ ID NO: 242) |
| X1866 | 5′-CTGGTGCAGCGCGAACTTCATGTTCTGCAT |
| | GTCC-3′ |
| | (SEQ ID NO: 243) |
| X1990 | 5′-TCAAGTTTGCGATTCATATGCTCCGTGCAA |
| | CAGGG-3′ |
| | (SEQ ID NO: 244) |
| X1991 | 5′-ATATGAATCGCAAACTTGATGTTGTGATTA |
| | AC-3′ |
| | (SEQ ID NO: 245) |
| X1867 | 5′-GTGGCTTTCGCGCTGCAACTTGTTAACGGC |
| | GG-3′ |
| | (SEQ ID NO: 246) |
| X1868 | 5′-GTTGCAGCGCGAAAGCCACGTTGTGGCCAC |
| | ATAACCC-3′ |
| | (SEQ ID NO: 247) |
| X1601 | 5′-GGCAAAACCTGAAATACGCGCGCGGTATTC |
| | TGG-3′ |
| | (SEQ ID NO: 248) |
| X1602 | 5′-CCAGAATACCGCGCGCGTATTTCAGGTTTT |
| | GCC-3′ |
| | (SEQ ID NO: 249) |
| X1903 | 5′-FAM-CCCCTTATTAGCGTTTGCCC-3′ |
| | (SEQ ID NO: 250) |
| X2362 | 5′TGCCAACGCGGGACGTGGCCAGCCCGTAGA |
| | ATGTTTTGTATTTGCATCATTTCATTTTCT |
| | CCCGCGATAT-3′ |
| | (SEQ ID NO: 251) |
| X2363 | 5′-FAM-ATATCGCGGGAGAAAATGAA-3′ |
| | (SEQ ID NO: 252) |
| X2364 | 5-CGGGTCACATCTTGTGCCCGAAGGATGTCA |
| | CGGATGTTTAGTTTGTCCATGGTTCAATCT |
| | CGCTCCAAGG-3′ |
| | (SEQ ID NO: 253) |
| X2365 | 5′-FAM-CCTTGGAGCGAGATTGAACC-3′ |
| | (SEQ ID NO: 254) |
| X2366 | 5′-GAGGCCGGTGCCTGGTCCACCGGGCGTGCG |
| | AAGTACAGGTAGGGGGTCACTTCGCGGCCT |
| | GCCCCAACAG-3′ |
| | (SEQ ID NO: 255) |
| X2367 | 5′-FAM-CTGTTGGGGCAGGCCGCGAA-3′ |
| | (SEQ ID NO: 256) |
| X2368 | 5′-TGTCCGCCTGAGAAGTCGATCGGGTAGGCG |
| | AGGTATAGCGTCTTGGTCACAGCTTGACCG |
| | GTCCTTTCGT-3′ |
| | (SEQ ID NO: 257) |
| X2369 | 5′-FAM-ACGAAAGGACCGGTCAAGCT-3′ |
| | (SEQ ID NO: 258) |
| X2426 | 5′-TCCTTCCCAACGCGCGGGTCCAGAATCTTA |
| | ACGTCGATTTTTTTCATCATAACGGGTCAC |
| | GATCTCGTCG-3′ |
| | (SEQ ID NO: 259) |
| X2427 | 5′-FAM-CGACGAGATCGTGACCCGTT-3′ |
| | (SEQ ID NO: 260) |
| X1648 | 5′-FAM-GGTATTAGCGCGCTCG-3′ |
| | (SEQ ID NO: 261) |
| X1649 | 5′-GCGTTGTTCCGGAAGTCGAGCGCGCTAATA |
| | -3′ |
| | (SEQ ID NO: 262) |
| X1459 | 5′-FAM-CCCCTTATTAGCGTTTGCC |
| | AAAAAAAAAAAAAAAAAAAAAAAAA-3′ |
| | (SEQ ID NO: 263) |
| X1904 | 5′-TTTTTTTTTTTTTTTTTTTTTTT |
| | GGGCAAACGCTAATAAGG-3′ |
| | (SEQ ID NO: 264) |
| X2364 | 5′-CGGGTCACATCTTGTGCCCGAAGGATGTCA |
| | CGGATGTTTAGTTTGTCCATGGTTCAATCT |
| | CGCTCCAAGG-3′ |
| | (SEQ ID NO: 265) |
| X2365 | 5′-FAM-CCTTGGAGCGAGATTGAACC-3′ |
| | (SEQ ID NO: 266) |
| X2586 | 5′-CGGGTCZCZTCTTGTGCCCGZZGGZTGTCZ |
| | CGGZTGTTTZGTTTGTCCZTGGTTCZZTCT |
| | CGCTCCZZGG-3′ |
| | (SEQ ID NO: 267) |
| X2587 | 5′-FAM-CCTTGGZGCGZGZTTGZZCC-3′ |
| | (SEQ ID NO: 268) |
| X1929 | 5′-CCCCCCCCCCCCCCCCCCCCCCCCGGGCAA |
| | ACGCTAATAAGG-3′ |
| | (SEQ ID NO: 269) |

1. A method for the production of a DNA molecule comprising 2-amino-2′-deoxyadenosine (dZ) instead of deoxyadenosine (dA) comprising the steps of:
(i) Providing a template DNA molecule to be replicated;
(ii) Providing dNTPs and wherein said dNTPs include 2-amino-2′-deoxyadenosine-triphosphate (dZTP);
(iii) Providing a polymerase, wherein the polymerase is characterized by the following features:
(a) it has an amino acid sequence which is at least 50% identical to the amino acid sequence shown in any one of SEQ ID NOs: 1 to 233;
(b) it does not show 5′-exonuclease activity;
(c) it accepts 2-amino-2′-deoxyadenosine 5′-triphosphate (dZTP) as a substrate; and
(d) it does not accept deoxyadenosine 5′-triphosphate (dATP) as a substrate; and
(iv) Incubating the components of (i) to (iii).

2. The method of claim 1, wherein the polymerase is furthermore characterized by the feature that it shows 3′-exonuclease activity.

3. The method of claim 1, wherein the polymerase is furthermore characterized by the feature that it does not show 3′-exonuclease activity.

4. The method of any one of claims 1 to 3 which furthermore comprises the step of recovering the produced DNA molecule comprising 2-amino-2′-deoxyadenosine (dZ).

5. The method of any one of claims 1 to 4 which is carried out in vitro.

6. The method of any one of claims 1 to 4 which is carried out in vivo.

7. Use of a polymerase as defined in any one of claims 1 to 3 for the production of a DNA molecule comprising 2-amino-2′-deoxyadenosine (dZ) instead of deoxyadenosine (dA).

8. A recombinant nucleic acid molecule comprising a nucleotide sequence encoding a polymerase wherein the polymerase is characterized by the following features:
(a) it has an amino acid sequence which is at least 50% identical to the amino acid sequence shown in any one of SEQ ID NOs: 1 to 233;
(b) it does not show 5′-exonuclease activity;
(c) it accepts 2-amino-2′-deoxyadenosine 5′-triphosphate (dZTP) as a substrate; and
(d) it does not accept deoxyadenosine 5′-triphosphate (dATP) as a substrate.

9. The recombinant nucleic acid molecule of claim 8, wherein the polymerase is furthermore characterized by the feature that it shows 3′-exonuclease activity.

10. The recombinant nucleic acid molecule of claim 8, wherein the polymerase is furthermore characterized by the feature that it does not show 3′-exonuclease activity.

11. The recombinant nucleic acid molecule of any one of claims 8 to 10, wherein the nucleotide sequence encoding the polymerase is operatively linked to a heterologous promoter sequence.

12. A vector comprising the recombinant nucleic acid molecule of any one of claims 8 to 11.

13. A host cell comprising the recombinant nucleic acid molecule of any one of claims 8 to 11 or the vector of claim 12.

14. A composition comprising a recombinant nucleic acid molecule of any one of claims 8 to 11, a polymerase as defined in any one of claims 8 to 10, a vector of claim 12 or a host cell of claim 13 and dZTP (and optionally further dNTPS like dTTP, dGTP and dCTP).

15. A kit comprising a recombinant nucleic acid molecule of any one of claims 8 to 11, a polymerase as defined in any one of claims 8 to 10, a vector of claim 12 or a host cell of claim 13 and dZTP (and optionally further dNTPS like dTTP, dGTP and dCTP)

16. A polymerase encoded by the nucleic acid molecule of 10.

17. The polymerase of claim 16 characterized in that it contains in its amino acid sequence a substitution at the position which corresponds to position D424 of the amino acid sequence of E. coli pol I (SEQ ID NO: 234), preferably a substitution by alanine.
